(19) United States
(12) Patent Application Publication
(10) Pub. No.: US 2024/0158770 A1
(43) Pub. Date: May 16, 2024

(54) PDE5A DESTABILIZING DOMAINS

(71) Applicant: The Board of Trustees of the Leland Stanford Junior University, Stanford, CA (US)

(72) Inventors: Thomas J. Wandless, Menlo Park, CA (US); Ling-Chun CHEN, Stanford, CA (US); Yusaku MIYAMAE, Ibaraki (JP)

(21) Appl. No.: 18/391,409

(22) Filed: Dec. 20, 2023

Related U.S. Application Data
(62) Division of application No. 16/625,644, filed on Dec. 20, 2019, now Pat. No. 11,891,634.
(60) Provisional application No. 62/524,384, filed on Jun. 23, 2017.

(51) Int. Cl. C12N 9/16; C07K 14/435; C12N 15/10; C12N 15/62

(52) U.S. Cl. CPC C12N 9/16; C07K 14/435; C12N 15/1055; C12N 15/62; C12Y 301/04017; C07K 2319/70

(57) ABSTRACT

Disclosed herein are systems, methods, and compositions for rapidly and reversibly destabilizing a target protein in vitro or in vivo, in the presence or absence of a cell-permeable, synthetic molecule or ligand.

### CROSS-REFERENCE TO RELATED APPLICATIONS

This application is a divisional application of U.S. application Ser. No. 16/625,644, filed Dec. 20, 2019, allowed, which is a National Stage Entry of PCT/US2018/039096, filed Jun. 22, 2018, which claims priority to 62/524,384, filed on Jun. 23, 2017, each of which is incorporated herein by reference in its entirety.

### STATEMENT REGARDING FEDERALLY SPONSORED RESEARCH OR DEVELOPMENT

This section intentionally left blank.

### REFERENCE TO THE SEQUENCE LISTING

This application contains a Sequence Listing which has been filed electronically in ASCII format and is hereby incorporated by reference in its entirety. The ASCII copy, created on Jun. 22, 2018, is named 091511-0624_8285_WO00_SL.txt and is 213,645 bytes in size.

### TECHNICAL FIELD

Rapid and reversible methods for destabilizing specific proteins using cell permeable, synthetic molecules are described. Variants of the human PDE5A proteins that are rapidly and constitutively degraded in the cells confer instability to the fusion proteins. Addition of a ligand that binds to the variant destabilizing domain prevents protein degradation, allowing the biological function of the fusion protein to be studied in detail.

### BACKGROUND

The ability to control the abundance of a specific protein in cells represents a powerful approach to interrogating complex biological behavior and in gene therapy. One of the most well-established ways to modulate protein activity is to knockdown the corresponding gene-of-interest either by targeting its precursor DNA or RNA molecules. While recent advances in genome editing tools have significantly improved the efficiency of perturbing specific genes, such efforts can be laborious and are not readily reversible. Alternatively, RNAi allows researchers to more quickly assess the effects of gene silencing; however, this approach is often plagued by incomplete knockdown, off-target specificity, and other nonspecific interactions. Because each of these approaches modulates a protein indirectly, they ultimately suffer from long experimental delays, given that the previously transcribed and synthesized protein molecules must be degraded before effects can be observed.

To circumvent the challenges modulating gene-of-interest, approaches that directly control protein level using cell permeable small molecules have been developed. These approaches directly recruit enzymes involved in the ubiquitin-proteasome system (UPS) to the protein of interest (POI), thereby promoting its degradation. One approach involves a cell-permeable ligand that is used in conjunction with a single genetically encoded domain to regulate any protein of interest, to which the domain is genetically fused. Mutants of domains (proteins) are engineered to be metabolically unstable in the absence of their high affinity ligand and are referred to as the destabilizing domains (DDs) (Stankunas, K., et al., (2003). Mol. Cell 12, 1615-1624; Banaszynski, L. A., et al., (2006) Cell, 126(5): 995-1004; reviewed in Banaszynski, L. A., and Wandless, T. J. (2006) Chem. Biol. 13, 11-21; Iwamoto, M., et al. (2010). Chem Biol. 17(9):981-8; Egeler, E. L. et al. (2011). J Biol Chem. 286(36):31328-36; and Rakhit R, Navarro R, Wandless T J (2014) Chem Biol. September 18; 21(9):1238-52; Navarro, R. et al. (2016) ACS Chem Biol. 11(8): 2101-2104). The instability of a DD, conferred to any fused partner protein results in degradation of the entire protein by the proteasome. The high affinity ligand binds to and stabilizes the DD in a dose dependent manner. The genetic fusion of the DD to the protein of interest ensures specificity, and small-molecule control confers reversibility and dose-dependence to protein stability and function.

A system as herein described, is a ligand regulated protein stability system, with the Destabilizing Domains containing protein stability systems as the prototype model. Controlling protein function using protein stability systems described herein is a more attractive approach than targeting precursor DNA or mRNA, as targeting of protein is rapid and is not limited by the intrinsic half-life of targeted proteins.

Provided are novel protein domains, in particular, destabilizing domains derived from human cGMP-specific Phosphodiesterase type 5A (PDE5A), particularly, the catalytic domain of human PDE5A, that displays small molecule dependent ligand stability; and the protein stability systems comprising such DDs. Methods for conditionally stabilizing proteins using the same are also provided.

### BRIEF SUMMARY

Provided herein are novel protein domains displaying ligand dependent stability. Such protein domains are called destabilizing domains (DDs). In the absence of its binding ligand, the DD is destabilizing and causes degradation of a payload fused to the DD (e.g., a protein of interest (POI), while in the presence of its binding ligand, the fused DD and payload can be stabilized and its stability is dose dependent.

In some aspects, conditional protein stability systems are provided, the systems comprising a protein of interest fused in-frame to a single-protein, ligand dependent destabilization domain derived from a region or portion of human PDE5A. The destabilization domain may be derived from the catalytic domain of PDE5A and/or the GAF domain; and may include one or more amino acid substitutions selected from E535D, E536G, Q541R, K555R, F559L S560G, F561L, F564L, F564S, V585A, N587S, K591E, 1599V, K604E, K608E, N609H, K630R, K633E, N636S, 1648V, N661S, S663P, L675P, Y676D, Y676N, C677R, H678R, D687A, T711A, T712S, D724N, L738H, N742S, F744L, L746S, F755L, A762S, D764V, D764N, D764G, S766F, K795E, L797F, I799T, L804P, T802P, S815C, M816A, M816T, I824T, C839S, F840S, and K852E. The PDE5A derived DDs may also contain additional substitutions such as Q589R. In some embodiments, the conditional protein stability system comprises a destabilizing domain selected from the group of amino acid sequences identified by SEQ ID NOs. 19-35 (encoded by SEQ ID NOs. 36-52) and SEQ ID NOs. 66-69 (encoded by SEQ ID NO. 70-73).

In some embodiments, the ligand may be Sildenafil, Vardenafil, Tadalafil, Avanafil, Lodenafil, Mirodenafil, Udenafil, Benzamidenafil, Dasantafil, or Beminafil.

In one embodiment, cells comprising nucleic acids encoding a fusion protein comprising a protein of interest fused in frame to a PDE5A derived destabilizing domain are provided. In some embodiments, the cells are in an organism. In another aspect, a kit of parts comprising PDE5A derived DDs is provided.

Methods for conditionally stabilizing a protein of interest, using the DDs, systems and compositions are also provided.

In addition to the exemplary aspects and embodiments described above, further aspects and embodiments will become apparent by reference to the drawings and by study of the following descriptions.

### BRIEF DESCRIPTION OF THE DRAWINGS

FIG. 1 illustrates a method for conditionally controlling protein stability. A genetic fusion of a destabilizing domain (DD) to a protein of interest (PO) results in degradation of the entire fusion protein. Addition of a ligand for the destabilizing domain protects the fusion protein from degradation.

FIG. 2 is a western blot depicting the ligand dependent stabilization of PDE5A, C terminal fusion proteins.

FIG. 3 is a western blot depicting the effect of HA tag on the stability of PDE5A mutants.

FIG. 4A and FIG. 4B show Sildenafil dose dependent stabilization of PDE5A mutants.

### DETAILED DESCRIPTION

### I. Introduction

Techniques for modulating transcription of DNA and RNA expression provide powerful tools for studying specific genes and their biological function. For example, the tet/dox, and Cre/lox systems have been widely used to target gene expression at the transcriptional level (Ryding A. D. S. et al. (2001), J. Endocrinol. 171:1-14) and RNA interference provides a method to achieve post-transcriptional gene silencing Fire, A. et al. (1998) Nature 391:806-811; Medema, R. H. (2004) Biochem. J. 380:593-603; Raab, R. M. and Stephanopoulos, G. (2004) Biotechnology & Bioengineering 88:121-132).

Techniques have also been developed to regulate proteins on a post-translational level. Experimental methods have been developed to regulate protein stability and function rapidly and reversibly using protein domains that are conditionally stable in cultured cell or living animals. Such methods are often controlled by the binding of a small molecule ligand (Baker, M. (2012) Nat. Methods 9, 443-447). Methods to conditionally regulate protein abundance in cells are useful to biologists to study a protein's function(s) in complex biological systems. However, methods for regulating protein function directly are limited, especially in mammalian cells. Inhibitors or activators of particular proteins have been identified, and often take the form of cell permeable small molecules. Many of these molecules have found widespread use as biological probes, often because the speed, dosage-dependence, and reversibility of their activities, which complement methods for genetically modulating (Schreiber, S. L. (2003) Chem. & Eng. News 81:51-61). However, these inhibitors or activators are often promiscuous, affecting several proteins rather than a specific protein (Davies, S. P. et al. (2000) Biochem. J. 351:95-105; Bain, J. et al. (2003) Biochem. J. 371:199-204; Godl, K. et al. (2003) Proc. Natl. Acad. Sci. U.S.A. 100:15434-15439; Tan, D. S. (2005) Nat. Chem. Biol. 1:74-84; Mayer, T. U. et al. (1999) Science, 286:971-974).

A method by which specific kinases can be inhibited using a small molecule modulator has also been developed (Bishop, A. C. et al. (1998) Current Biology 8:257-266). This method involved mutating a protein of interest, typically replacing a large conserved residue in the active site with a smaller residue, such as glycine or alanine. Specificity is achieved by chemically modifying a promiscuous inhibitor to include a bulky side-chain substituent (e.g. R-group), which fills the corresponding cavity in the binding site of the modified protein of interest, while preventing the productive interactions with other kinases. While this so called “bump-hole” approach has been successful both in cultured cells and in mice Bishop, A. C. et al. (2000) Nature 407:395-401; Wang, H. et al. (2003) Proc. Natl. Acad. Sci. U.S.A. 100:4287-4292, Chen, X. et al. (2005) Neuron 46:13-21), it appears to be limited to ATPases and GTPases. Additional methods are required to probe the function of a wider variety of proteins.

Alternative strategies to perturb protein function by exploring existing cellular processes have been devised. For example, a method has been developed for controlling protein function based on the importance of certain N-terminal residues for protein stability (Bachmair, A. et al. (1986). Science 234:179-186).

Szostak and coworkers showed that a small peptide sequence could be fused to the N-terminus of a protein of interest to modulate protein stability (Park, E-C. et al. (1992) Proc. Natl. Acad. Sci. U.S.A. 89:1249-1252). Varshavsky and coworkers have further isolated a temperature sensitive peptide sequence that greatly reduced the half-life of dihydrofolate reductase (DHFR) at the non-permissive temperature (Dohmen, R. J. et al. (1994) Science 263:1273-1276). This approach has been used to study proteins in yeast (Labib, K. et al. (2000) Science 288:1643-1646; Kanemaki, M. et al. (2003) Nature 423:720-724).

Furthermore, dimeric small molecules have been engineered to conditionally target fusion proteins for degradation via E3 ligase or the proteasome, itself (Janse, D. M. et al. (2004) J. Biol. Chem. 279:21415-21420). However, these systems require either a prior knowledge of the high-affinity ligands that modulate the activity of a protein of interest or they are restricted to genetically engineered yeast strains.

An alternative approach for controlling protein function directly is to interfere with subcellular localization. For example, several methods have been developed to regulate protein localization using a small molecule by taking advantage of the FKBP-FRB ternary complex (Kohler, J. J. et al. (2003) Chem. Biol. 10:1303-1331; Inoue, T. et al. (2005) Nature Methods 2:415-418). Rapamycin and FK506 are potent, commercially available immunosuppressive agents, which are ligands of the FK506—binding protein (FKBP12, FKBP). Rapamycin also binds to FKBP-rapamycin-associated protein (FRAP). FRAP is also called the mammalian target of rapamycin (mTOR), rapamycin and FKBP target 1 (RAFT1), and FKBP-rapamycin-binding (FRB). Rapamycin binds to and inhibits FRAP/mTOR by interacting with its FRB domain to inhibit/delay G1 cell cycle progression in mammalian cells (see, e.g. Choi, J. et al. (1996) Science 273:239-42 and Vilella-Bach, M. et al. (1999) J. Biol. Chem. 274:4266-72. The FRB domain is required for FKBP-rapamycin-associated protein kinase activity and G1 progression. Fusions of proteins of interest can be made to either FKBP or to the FRP domain of FRAP/mTOR. Colocalization of the protein of interest is induced upon addition of rapamycin. Because rapamycin has inherent biological activity, researchers have developed a “bump-hole” strategy (similar to that described above), wherein rapamycin derivatives possessing large substituents at the FRB binding interface bind poorly to the wild-type FRB domain and thus the target FRAP/mTOR; binding is restored upon introduction of compensatory cavity-forming mutations in FRB. Specifically, a C20-methallyl-rapamycin derivative (MaRap) binds to a triple-mutated variant of FRB called FRB* (Liberles, S. D. et al. (1997) Proc. Natl. Acad. Sci. U.S.A. 94:7825-7830).

Recently, numerous applications have been described employing light responsive protein domains. The LOV (Light-Oxygen-Voltage) domains are part of plant photoreceptor proteins and detect blue light via a flavin cofactor (Herrou, J. et al. Nat. Rev. Microbiol. 9, 713-723 (2011)). The LOV2 domain of oat phototropin 1 (AsLOV2) holds a c-terminal alpha helix that is tightly bound to the LOV core domain in dark. Exposure to light results in unfolding of the helix. The AsLOV2 domain has been used to regulate certain protein activities by the steric inhibition of an effector protein or by restricting a specific protein conformation. (Wu, Y. I. et al. (2009) Nature 461, 104-108; Lee, J. et al. (2008) Science 322, 438-442; Strickland, D., et al. (2008) Proc. Natl. Acad. Sci. USA 105, 10709-10714). These methods are suitable for the reported engineered proteins but not generally applicable to other proteins. Alternatively, engineered AsLOV2 and other photosensory domains have been used to establish light mediated protein-protein interactions (Shimizu-Sato, S., et al. (2002) Nat. Biotechnol. 20, 1041-1044; Levskaya, A., et al. (2009) Nature 461, 997-1001; Yazawa, M., et al. (2009) Nat. Biotechnol. 27, 941-945; Kennedy, M. J. et al. (2010) Nat. Methods 7, 973-975; Strickland, D. et al. (2012) Nat. Methods 9, 379-384; Wang, X., et al. (2012) Nat. Methods 9, 266-269; Polstein, L. R. et al. (2012) J. Am. Chem. Soc. 134, 16480-16483; Lungu, O. I., et al. (2012) Chem. Biol. 19, 507-517; Zhou, X. X. et al. (2012) Science 338, 810-814). Thus, translocation strategies to the cell membrane or the nucleus can allow location specific protein activity and light induced gene expression systems respectively. These technologies may be useful in some cases, but they often lack the ability to control protein levels once present in the cells, and none of the existing methods developed so far is suitable for fast and reversible regulation of protein levels. Furthermore, while the aforementioned methods for regulating protein function directly are noteworthy, a need remains for a convenient, general method for regulating protein function, particularly a method that does not require the interaction of multiple proteins. Regulation of protein stability in cells in a more spatial or temporal manner is also desirable.

Building on the FRB* domain system, Banaszynski et al., developed a cell permeable ligand system of mutants of FKBP12 which are engineered to be unstable in the absence of a high affinity ligand, Shield-1 (Banaszynski et al., Cell. (2006); 126:995-1004). They termed these unstable domains, destabilizing domains (DDs). The FKBP/shield-1 tuning system has been successfully used in several studies to control target proteins. For example, Dettwier et al., fused FKBP to tune the express of NADPH P450 oxidoreductase (POR) (Dettwier et al., PLoS One, 2014, 9(11): e113540). The FKBP DD-shield system has been used in cell lines, transgenic mice, protozoan Entamoeba histolytica, the flatworm Caenorhabditis elegans, the medaka, and transgenic xenografts to investigate the activity of a protein of interest (Maynard-Smith et al., (2007), J Biol Chem. 282(34): 24866-24872; Liu et al., (2014) Int J Parasitol., 44(10):729-735; Cho et al., (2013) PLoS One., 8(8): e72393; Banaszynski et al. (2008) Nat Med, 14(10):1123-1127; Rodriguez and Wolfgang, (2011) Chem Biol., 19(3):391-398; and Froschauer et al., (2015) PLoS One, 10(7): e0131252), for iPSC reprogramming (Sui et al., (2014) Stem cell Reports., 2(5): 721-733).

Other DD ligand pairs include estrogen receptor domains which can be regulated by several estrogen receptor antagonists (Miyazaki et al., (2012) J Am Chem. Soc, 134(9): 3942-3945), and fluorescent destabilizing domain (FDD) derived from bilirubin-inducible fluorescent protein, UnaG. A FDD and its cognate ligand bilirubin (BR) can induce degradation of a protein fused to the FDD (Navarro et al. (2016) ACS Chem Biol, June 6, Epub). Other known DDs and their applications in protein stability include those described in U.S. Pat. Nos. 8,173,792 and 8,530,636, the contents of which are each incorporated herein by reference in their entirety.

In an orthogonal approach, the destabilizing domains of the bacterial dihydrofolate reductase (ecDHFR) were explored. (Iwamoto et al. (2010) Chem Biol., 17(9):981-988; and Tai et al. (2012), PLoS One. 7(9): e46269). Numerous inhibitors of DHFR have been developed as drugs and one such inhibitor Trimethoprim (TMP), inhibits ecDHFR much more potently than mammalian DHFR providing specificity to the interaction (Iwamoto, et al., (2010) Chem Biol. September 24; 17(9): 981-988).

The present findings expand upon the technology of conditional protein stability systems by utilizing destabilizing domains derived from human PDE5A protein. The destabilization and stabilization of a protein of interest can be controlled by PDE5A derived DDs having stabilizing or destabilizing properties and their ligands e.g. Sildenafil and Vardenafil which bind to such protein domains. The presence and/or absence of the ligand can conditionally stabilize the protein of interest that is genetically fused to the protein of interest.

### II. Compositions

Described herein are compositions, systems and methods for modulating the stability and function of proteins rapidly and reversibly, in vitro and in vivo, through the administration of cell-permeable small molecules to cultured cells or living animals. The coding sequence for a protein of interest (POI) is genetically fused to a sequence encoding a stability-affecting protein domain capable of interacting with a small-molecule ligand, the presence, absence, or amount of which ligand modulates the stability of the fusion protein. These compositions, systems and methods are designed to provide (1) protein domains in the form of cDNA constructs that, when fused to any gene-of-interest, can be used by investigators to degrade proteins-of-interest; and (2) cell-permeable small molecules that bind to and stabilize the destabilizing domains (DD), thereby restoring the function of the protein-of-interest. A feature of the conditional system is that it is a “single ligand domain” system, which minimizes the number of components in the system and the complexity of the system.

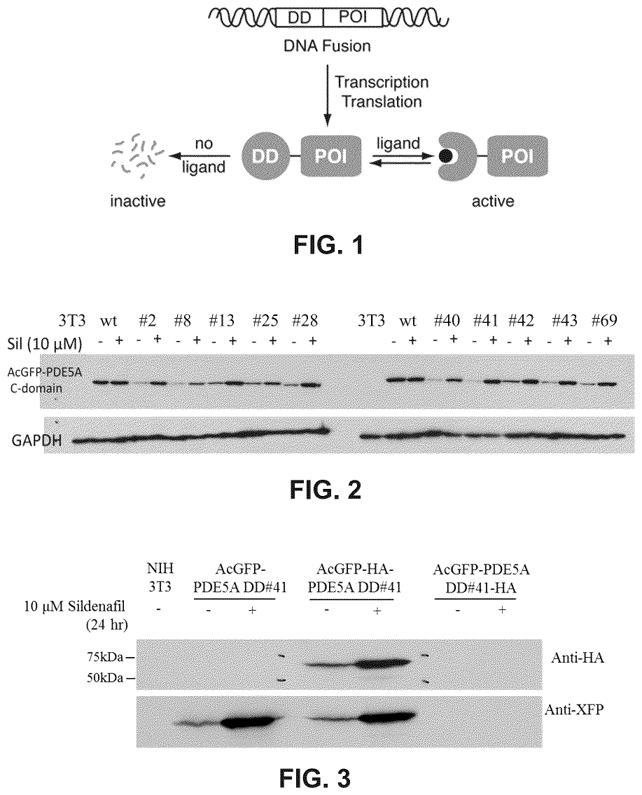

In some embodiments, provided are ligand-regulated conditional protein stability systems which enable the stability of a protein of interest to be modulated (i.e. manipulated or controlled), following its expression. In conditional protein stability systems described herein, stability is modulated by adding an amount of a stabilizing ligand, which binds to a fusion protein containing a destabilizing protein domain fused to a POI, thereby stabilizing the fusion protein and allowing the POI to function in the cell. As used herein, “POI” refers to any protein, or functional fragment or derivative, thereof, that one skilled in the art wishes to study, or for which, one desires to conditionally destabilize and regulate the degradation of the protein functional fragment or derivative thereof. Described herein are ligand regulated protein stability systems and destabilizing protein domains comprising PDE5A derived destabilizing domains. The overall architecture of the ligand regulated protein stability system is presented in FIG. 1. Introducing a fusion protein consisting of the DD and the protein interest into a cell results in the expression of the protein. The stability of the fusion protein is modulated upon administration of the ligand.

In some embodiments, stability of the fusion protein is increased upon administration of the ligand. This strategy is referred to as the “drug-on” in that the stabilizing ligand must be present for the expression of the desired fusion protein. However, if the POI exhibits a dominant negative phenotype, the system may effectively be “drug-off” in that removal of the stabilizing ligand is required for the expression of the desired fusion protein. In this embodiment, addition of a stabilizing ligand results in the stabilization of the fusion protein and loss of function of the target protein. A similar situation may exist if a constitutively active variant of a protein e.g. an oncogene, was placed under the control of a ligand responsive fusion protein, wherein the addition of ligand (rather than its withdrawal) triggers the event of interest and ensures the specificity of the conditional stabilization. This may result in a decrease in the stability of the fusion protein upon administration of the ligand.

Preferred destabilizing domains direct the degradation of a fusion protein in the absence of a stabilizing small molecule. Preferred destabilization domains reduce the amount of fusion protein present in a cell to less than 20%, less than 10%, less than 5%, less than 2% or even less than 1% of the amount of the fusion protein present upon the addition of the stabilizing ligand, or compared to the amount of the naturally-occurring POI (i.e. the native protein, not a fusion protein). In this context, the naturally occurring POI is deleted or disrupted in the genome of the cells or animal in which the conditional protein stability system is used or replaced by a DNA encoding the fusion protein. In this manner, the only source of the POI is the conditionally stabilized fusion protein, allowing its function to be studied in the absence of interfering wildtype/naturally-occurring protein.

In some embodiments, methods for modulating protein, expression, function or level by measuring the stabilization ratio are provided. As used herein, the stabilization ratio may be defined as the ratio of expression, function or level of a protein of interest in response to the ligand to the expression, function or level of the protein of interest in the absence of the ligand specific to the destabilizing domain. In some aspects, the stabilization ratio is at least 1, such as by at least 1-10, 1-20, 1-30, 1-40, 1-50, 1-60, 1-70, 1-80, 1-90, 1-100, 20-30, 20-40, 20-50, 20-60, 20-70, 20-80, 20-90, 20-95, 20-100, 30-40, 30-50, 30-60, 30-70, 30-80, 30-90, 30-95, 30-100, 40-50, 40-60, 40-70, 40-80, 40-90, 40-95, 40-100, 50-60, 50-70, 50-80, 50-90, 50-95, 50-100, 60-70, 60-80, 60-90, 60-95, 60-100, 70-80, 70-90, 70-95, 70-100, 80-90, 80-95, 80-100, 90-95, 90-100 or 95-100.

The position of the POI with respect to the DD, within the fusion protein to achieve optimal DD regulation. In some embodiments, the POI may be fused to the N terminus of the DD. In another embodiment, the POI may be fused to the C terminus of the DDs. In some embodiments, the fusion protein may include more than one POIs fused to one or more DDs.

### Destabilizing Domains

As used herein, the term “destabilizing domains” (DDs), refers to protein domains that are unstable and degraded in the absence of ligand, but whose stability is rescued by binding to a high affinity cell-permeable ligand. Destabilizing domains (DDs) can be fused to a target protein of interest (POI) and can convey its destabilizing property to the protein of interest, causing protein degradation. The presence, absence or an amount of a small molecule ligand that binds to or interacts with the DD, can, upon such binding or interaction modulate the stability of the payload(s) and consequently the function of the payload. A protein domain with destabilizing property (e.g. a DD) is used in conjunction with a cell-permeable ligand to regulate any protein of interest when it is fused with the destabilizing domain. DDs render the attached protein of interest unstable in the absence of a DD-binding ligand such that the protein is rapidly degraded by the ubiquitin-proteasome system of the cell. However, when a specific small molecule ligand binds its intended DD as a ligand binding partner, the instability is reversed and protein function is restored. The conditional nature of DD stability allows a rapid and non-perturbing switch from stable protein to unstable substrate for degradation. Such a destabilization domain may or may not require the interaction of another protein for modulating stability of the POI. In some embodiments, the ligand regulated destabilizing domain merely requires exposure to a small molecule ligand, and does not require the formation of a ternary complex, as does the FKBP-rapamycin-FRB complex. An exemplary species is a “single-domain”, ligand dependent destabilization domain, wherein the single polypeptide comprises only a single domain (i.e. folded structure or functional unit as determined by X-ray crystallography, protease digestion, and/or computer modelling).

Due to its reversibility, specificity and the fast and easy regulation on protein level, the post-transcriptional tuning system provides a useful system for gene regulation. Furthermore, the regulation may be dose-dependent, thereby altering the protein-turnover rate to transform a short-lived or no detectable protein into a protein that functions for a precisely controlled period of time (Iwamoto et al., Chem. Biol. 2010, 17: 981-988).

Candidate destabilizing domain sequence identified from protein domains of known wildtype proteins (as a template) may be mutated to generate libraries of mutants based on the template candidate domain sequence. Libraries of mutants may be generated by any methods known in the art including error prone polymerase chain reaction and nucleotide analog mutagenesis. Destabilizing domains identified using random mutagenesis may be used to identify structural properties of the candidate DDs that may be required for destabilization, which may then be used to further generate libraries of mutations using site directed mutagenesis.

### Human PDE5A Derived DDs

In some embodiments, the DDs of the present compositions and systems are derived from human PDE5A (cGMP-specific phosphodiesterase type 5A) protein. PDE5A is a cGMP selective phosphodiesterase and a member of the cyclic nucleotide phosphodiesterase family. PDE5A specifically hydrolyzes cGMP to 5′GMP thereby regulating intracellular concentrations of cyclic nucleotides, a process that is important for smooth muscle relaxation in the cardiovascular system.

In some embodiments, the PDE5A derived destabilizing domains may be derived from variants, and/or isoforms of PDE5A. Three isoforms of PDE5A namely, PDE5A, Isoform 1, PDE5A Isoform 2, and PDE5A Isoform 3 have been identified. These isoforms differ at their N terminal regions, and have unique first exons followed by a common sequence of 823 amino acids. Accordingly, PDE5 derived DDs may be derived from PDE5A, Isoform 1 (SEQ ID NO. 1; encoded by the nucleotide sequence of SEQ ID NO. 2); PDE5A Isoform 2 (SEQ ID NO. 3; encoded by SEQ ID NO. 61 or nucleotides 113-2614 of the nucleotide sequence of SEQ ID NO. 4) and/or PDE5A Isoform 3 (SEQ ID NO. 5; encoded by SEQ ID NO. 62 or nucleotides 95-2566 of the nucleotide sequence of SEQ ID NO. 6).

All PDE5A isoforms contain a catalytic domain that is located near the C terminus of the protein and is relatively selective for cGMP as a substrate at physiological levels. The substrate binding site is also the binding site for several known PDE5 inhibitors such as Sildenafil, which have been utilized to treat cardiovascular diseases and erectile dysfunction. Towards the N terminus, 2 homologous GAF domains are located. One of the GAF domains, GAF-A contains a high affinity binding site for cGMP. Occupancy of this domain by cGMP is known to cause activation of the catalytic domain. Moreover, the affinity of this site for cGMP is increased by cGMP-dependent protein kinase-mediated phosphorylation of serine 92. In another embodiment, the PDE5A derived DD may comprise the catalytic domain of PDE5A, spanning from amino acid position 535 to position 860 of UniProt ID: 076074 (SEQ ID NO. 1), as represented in SEQ ID NO. 7 (encoded by SEQ ID NO. 8). In addition to the catalytic domain, PDE5A derived DDs may also comprise one or more GAF domains and/or the C terminal portion that extends beyond the catalytic domain. In one embodiment, the PDE5A derived DD may consist of amino acids from position 535 to position 875 of SEQ ID NO. 1. In another embodiment, the PDE5A derived DD may consist of amino acids from position 466 to 875 or position 420 to 875 of SEQ ID NO. 1.

The destabilization domains described herein may also include amino acid and nucleotide substitutions that do not affect stability, including conservative, non-conservative substitutions and or polymorphisms. In one embodiment, the PDE5A derived DD may contain a polymorphism at residue 589 of SEQ ID NO. 1, wherein the DD may comprise an arginine residue instead of a glutamine residue and is denoted as Q589R. Additional polymorphisms may include glycine to aspartate substitution at residue 36 of SEQ ID NO. 1, denoted as G36D, and valine to alanine substitution at position 93 of SEQ ID NO. 1, denoted as V93A. In some embodiments, the PDE5 DD may comprise all three polymorphisms, G36D, V93A, Q589R. These polymorphisms are represented in GenBank Accession Number AB527373.1 consisting of the amino acid sequence of SEQ ID NO. 9, (encoded by SEQ ID NO. 10), wherein the annotation of amino acid position and mutation in “G36D, V93A, Q589R” is with respect to SEQ ID NO. 1. The catalytic domain of PDE5A with the Q589R polymorphism is represented by the amino acid sequence of SEQ ID NO. 11, encoded by the nucleotide sequence of SEQ ID NO. 12. PDE5A derived DDs with Q589R polymorphism described herein may also include amino acids 535-875 of PDE5A with Q589R polymorphism with amino acid sequence of SEQ ID NO. 13 (encoded by SEQ ID NO. 63). In some embodiments the nucleotide sequence of “535-875 of PDE5A” may include a stop codon (by SEQ ID NO. 14). In some embodiments the PDE5A derived DDs may include amino acids 466-875 of PDE5A comprising the Q589R polymorphism, and with amino acid sequence of SEQ ID NO. 15 (encoded by SEQ ID NO. 64). In some embodiments, the nucleotide sequence of “466-875 of PDE5A” may include a stop codon (SEQ ID NO. 16). In some embodiments, the PDE5A derived DDs may comprise amino acids 420-875 of PDE5A comprising Q589R polymorphism and with amino acid sequence of SEQ ID NO. 17 (encoded by SEQ ID NO. 65). In some embodiments, the nucleotide sequence of “420-875 of PDE5A” may include a stop codon (SEQ ID NO. 18).

According to the methods described herein, the inventors of the present disclosure identified several human PDE5A derived destabilizing mutations by random mutagenesis of the catalytic domain of PDE5A. The destabilization of the mutants in the absence of the binding ligand, Sildenafil was tested. Table 1 provides the PDE5A derived destabilizing mutations. The position number of the mutated amino acids listed in Table 1 is relative to the full length human PDE5A of SEQ ID NO. 1. In some embodiments, any of the PDE5A derived DDs may include a methionine at position 1 of the amino acid sequence.

TABLE 1
PDE5 DDs

| | | | Amino | Nucleic |
|---|---|---|---|---|
| | | | Acid | Acid |
| | Clone | | SEQ ID | SEQ ID |
| PDE5A mutant | NO. | Sequence | NO. | NO. |
| Methionine, | — | MEETRELQSLAAAVVPSAQTLKITDFSFSDFELSDLET | 19 | 36 |
| 535-860 of PDE5A | | ALCTIRMFTDLNLVQNFRMKHEVLCRWILSVKKNYR | | |
| (Q589R, K633E, | | KNVAYHNWRHAFNTAQCMFAALKAGEIQNKLTDLE | | |
| T712S, K852E, | | ILALLIAALSHDLDHRGVNNSYIQRSEHPLAQLYCHSI | | |
| K795E) | | MEHHHFDQCLMILNSPGNQILSGLSIEEYKTSLKIIKQ | | |
| | | AILATDLALYIKRRGEFFELIRKNQFNLEDPHQKELFL | | |
| | | AMLMTACDLSAITKPWPIQQRIAELVATEFFDQGDRE | | |
| | | REELNIEPTDLMNREKKNKIPSMQVGFIDAICLQLYE | | |
| | | ALTHVSEDCFPLLDGCRKNRQEWQALAEQQ | | |
| Methionine, | — | MEGTRELQSLAAAVVPSAQTLKITDFSFSDFELSDLE | 20 | 37 |
| 535-860 of PDE5A | | TALCTIRMFTDLNLVQNFRMKHEVLCRWILSVKKNY | | |
| (E536G, Q589R, | | RKNVAYHNWRHAFNTAQCMFAALKAGKIQNKLTD | | |
| C839S) | | LEILALLIAALSHDLDHRGVNNSYIQRSEHPLAQLYC | | |
| | | HSIMEHHHFDQCLMILNSPGNQILSGLSIEEYKTTLKII | | |
| | | KQAILATDLALYIKRRGEFFELIRKNQFNLEDPHQKE | | |
| | | LFLAMLMTACDLSAITKPWPIQQRIAELVATEFFDQG | | |
| | | DRERKELNIEPTDLMNREKKNKIPSMQVGFIDAICLQ | | |
| | | LYEALTHVSEDSFPLLDGCRKNRQKWQALAEQQ | | |
| Methionine, | — | MEETRELQSLAAAVVPSAQTLKITDFSFSDFELSDLET | 21 | 38 |
| 535-860 of PDE5A | | ALCTIRMFTDLNLVQSFRMKHEVLCRWILSVKKNYR | | |
| (N587S, Q589R, | | ENVAYHNWRHAFNTAQCMFAALKAGKIQNKLTDLE | | |
| K608E, N661 S, | | ILALLIAALSHDLDHRGVSNSYIQRSEHPLAQLYCHSI | | |
| D764V) | | MEHHHFDQCLMILNSPGNQILSGLSIEEYKTTLKIIKQ | | |
| | | AILATDLALYIKRRGEFFELIRKNQFNLEDPHQKELFL | | |
| | | AMLMTACVLSAITKPWPIQQRIAELVATEFFDQGDRE | | |
| | | RKELNIEPTDLMNREKKNKIPSMQVGFIDAICLQLYE | | |
| | | ALTHVSEDCFPLLDGCRKNRQKWQALAEQQ | | |
| Methionine, | — | MEETRELQSLAAAVVPSAQTLKITDFSFSDFELSDLET | 22 | 39 |
| 535-860 of PDE5A | | ALCTIRMFTDLNLVQNFRMKHEVLCRWILSVKKNYR | | |
| (Q589R, L675P, | | KNVAYHNWRHAFNTAQCMFAALKAGKIQNKLTDL | | |
| F755L) | | EILALLIAALSHDLDHRGVNNSYIQRSEHPLAQPYCH | | |
| | | SIMEHHHFDQCLMILNSPGNQILSGLSIEEYKTTLKIIK | | |
| | | QAILATDLALYIKRRGEFFELIRKNQFNLEDPHQKELL | | |
| | | LAMLMTACDLSAITKPWPIQQRIAELVATEFFDQGDR | | |
| | | ERKELNIEPTDLMNREKKNKIPSMQVGFIDAICLQLY | | |
| | | EALTHVSEDCFPLLDGCRKNRQKWQALAEQQ | | |
| 535-860 of | #2 | EETRELQSLAAAVVPSAQTLKITDFSFSDFELSDLETA | 23 | 40 |
| PDE5A (Q589R, | | LCTIRMFTDLNLVQNFRMKHEVLCRWILSVKKNYRK | | |
| D687A, D764N, | | NVAYHNWRHAFNTAQCMFAALKAGKIQNKLTDLEI | | |
| S815C) | | LALLIAALSHDLDHRGVNNSYIQRSEHPLAQLYCHSI | | |
| | | MEHHHFAQCLMILNSPGNQILSGLSIEEYKTTLKIIKQ | | |
| | | AILATDLALYIKRRGEFFELIRKNQFNLEDPHQKELFL | | |
| | | AMLMTACNLSAITKPWPIQQRIAELVATEFFDQGDRE | | |
| | | RKELNIEPTDLMNREKKNKIPCMQVGFIDAICLQLYE | | |
| | | ALTHVSEDCFPLLDGCRKNRQKWQALAEQQ | | |
| 535-860 of | #13 | EETRELQSLAAAVVPSAQTLKITDFSFSDFELSDLETA | 24 | 41 |
| PDE5A (Q589R, | | LCTIRMFTDLNLVQNFRMKHEVLCRWILSVKKNYRK | | |
| Y676D, L738H) | | NVAYHNWRHAFNTAQCMFAALKAGKIQNKLTDLEI | | |
| | | LALLIAALSHDLDHRGVNNSYIQRSEHPLAQLDCHSI | | |
| | | MEHHHFDQCLMILNSPGNQILSGLSIEEYKTTLKIIKQ | | |
| | | AILATDLALYIKRRGEFFEHIRKNQFNLEDPHQKELFL | | |
| | | AMLMTACDLSAITKPWPIQQRIAELVATEFFDQGDRE | | |
| | | RKELNIEPTDLMNREKKNKIPSMQVGFIDAICLQLYE | | |
| | | ALTHVSEDCFPLLDGCRKNRQKWQALAEQQ | | |
| 535-860 of | #28 | EETRELQSLAAAVVPSAQTLKITDFSFSDFELSDLETA | 25 | 42 |
| PDE5A (Q589R, | | LCTIRMFTDLNLVQNFRMEHEVLCRWILSVKKNYRK | | |
| K591E, N609H, | | HVAYHNWRHAFNTAQCMFAALKAGKIQNKLTDLEI | | |
| D764V) | | LALLIAALSHDLDHRGVNNSYIQRSEHPLAQLYCHSI | | |
| | | MEHHHFDQCLMILNSPGNQILSGLSIEEYKTTLKIIKQ | | |
| | | AILATDLALYIKRRGEFFELIRKNQFNLEDPHQKELFL | | |
| | | AMLMTACVLSAITKPWPIQQRIAELVATEFFDQGDRE | | |
| | | RKELNIEPTDLMNREKKNKIPSMQVGFIDAICLQLYE | | |
| | | ALTHVSEDCFPLLDGCRKNRQKWQALAEQQ | | |
| 535-860 of | #40 | EETRELQSLAAAVVPSAQTLKITDFSFSDFELSDLETA | 26 | 43 |
| PDE5A (Q589R, | | LCTIRMFTDLNLVQNFRMKHEVLCRWILSVKKNYRK | | |
| D764G) | | NVAYHNWRHAFNTAQCMFAALKAGKIQNKLTDLEI | | |
| | | LALLIAALSHDLDHRGVNNSYIQRSEHPLAQLYCHSI | | |
| | | MEHHHFDQCLMILNSPGNQILSGLSIEEYKTTLKIIKQ | | |
| | | AILATDLALYIKRRGEFFELIRKNQFNLEDPHQKELFL | | |
| | | AMLMTACGLSAITKPWPIQQRIAELVATEFFDQGDRE | | |
| | | RKELNIEPTDLMNREKKNKIPSMQVGFIDAICLQLYE | | |
| | | ALTHVSEDCFPLLDGCRKNRQKWQALAEQQ | | |
| 535-860 of | #41 | EETRELQSLAAAVVPSAQTLKITDFSLSDFELSDLETA | 27 | 44 |
| PDE5A (F561L, | | LCTIRMFTDLNLVQNFRMKHEVLCRWILSVKENYRK | | |
| Q589R, K604E, | | NVAYHNWRHAFNTAQCMFAALKAGKIQNKLTDLEI | | |
| D724N, L797F) | | LALLIAALSHDLDHRGVNNSYIQRSEHPLAQLYCHSI | | |
| | | MEHHHFDQCLMILNSPGNQILSGLSIEEYKTTLKIIKQ | | |
| | | AILATNLALYIKRRGEFFELIRKNQFNLEDPHQKELFL | | |
| | | AMLMTACDLSAITKPWPIQQRIAELVATEFFDQGDRE | | |
| | | RKEFNIEPTDLMNREKKNKIPSMQVGFIDAICLQLYE | | |
| | | ALTHVSEDCFPLLDGCRKNRQKWQALAEQQ | | |
| 535-860 of | #42 | DETRELQSLAAAVVPSAQTLRITDFSFSDSELSDLETA | 28 | 45 |
| PDE5A (E535D, | | LCTIRMFTDLNLVQNFRMKHEVLCRWILSVKKNYRK | | |
| K555R, F564S, | | NVAYHNWRHAFNTAQCMFAALRAGKIQNKLTDLEI | | |
| Q589R, K630R, | | LALLIAALSHDLDHRGVNNSYIQRSEHPLAQLYRHSI | | |
| C677R, N742S, | | MEHHHFDQCLMILNSPGNQILSGLSIEEYKTTLKIIKQ | | |
| I799T, M816A) | | AILATDLALYIKRRGEFFELIRKSQFNLEDPHQKELFL | | |
| | | AMLMTACDLSAITKPWPIQQRIAELVATEFFDQGDRE | | |
| | | RKELNTEPTDLMNREKKNKIPSAQVGFIDAICLQLYE | | |
| | | ALTHVSEDCFPLLDGCRKNRQKWQALAEQQ | | |
| 535-860 of | #43 | EETRELQSLAAAVVPSAQTLKITDFSFSDFELSDLETA | 29 | 46 |
| PDE5A (Q589R, | | LCTIRMFTDLNLVQNFRMKHEVLCRWILSVKKNYRK | | |
| N609H, Y676N, | | HVAYHNWRHAFNTAQCMFAALKAGKIQNKLTDLEI | | |
| A762S) | | LALLIAALSHDLDHRGVNNSYIQRSEHPLAQLNCHSI | | |
| | | MEHHHFDQCLMILNSPGNQILSGLSIEEYKTTLKIIKQ | | |
| | | AILATDLALYIKRRGEFFELIRKNQFNLEDPHQKELFL | | |
| | | AMLMTSCDLSAITKPWPIQQRIAELVATEFFDQGDRE | | |
| | | RKELNIEPTDLMNREKKNKIPSMQVGFIDAICLQLYE | | |
| | | ALTHVSEDCFPLLDGCRKNRQKWQALAEQQ | | |
| 535-860 of | #69 | EETRELQSLAAAVVPSAQTLKITDFSFSDFELSDLETA | 30 | 47 |
| PDE5A (Q589R, | | LCTIRMFTDLNLVQNFRMKHEVLCRWILSVKKNYRK | | |
| N636S, D687A, | | NVAYHNWRHAFNTAQCMFAALKAGKIQSKLTDLEI | | |
| D764N, S815C) | | LALLIAALSHDLDHRGVNNSYIQRSEHPLAQLYCHSI | | |
| | | MEHHHFAQCLMILNSPGNQILSGLSIEEYKTTLKIIKQ | | |
| | | AILATDLALYIKRRGEFFELIRKNQFNLEDPHQKELFL | | |
| | | AMLMTACNLSAITKPWPIQQRIAELVATEFFDQGDRE | | |
| | | RKELNIEPTDLMNREKKNKIPCMQVGFIDAICLQLYE | | |
| | | ALTHVSEDCFPLLDGCRKNRQKWQALAEQQ | | |
| 535-860 of | #19 | EETRELQSLAAAVVPSAQTLKITDFSFSDFELSDLETA | 31 | 48 |
| PDE5A (Q589R, | | LCTIRMFTDLNLVQNFRMKHEVLCRWVLSVKKNYR | | |
| I599V, T711A, | | KNVAYHNWRHAFNTAQCMFAALKAGKIQNKLTDL | | |
| F744L, L746S, | | EILALLIAALSHDLDHRGVNNSYIQRSEHPLAQLYCH | | |
| L804P) | | SIMEHHHFDQCLMILNSPGNQILSGLSIEEYKATLKIIK | | |
| | | QAILATDLALYIKRRGEFFELIRKNQLNSEDPHQKELF | | |
| | | LAMLMTACDLSAITKPWPIQQRIAELVATEFFDQGDR | | |
| | | ERKELNIEPTDPMNREKKNKIPSMQVGFIDAICLQLY | | |
| | | EALTHVSEDCFPLLDGCRKNRQKWQALAEQQ | | |
| 535-860 of | #35 | EETRELQSLAAAVVPSAQTLKITDFGFSDFELSDLETA | 32 | 49 |
| PDE5A (S560G, | | LCTIRMFTDLNLAQSFRMEHEVLCRWILSVKKNYRK | | |
| V585A, N587S, | | NVAYHNWRHAFNTAQCMFAALKAGKIQNKLTDLEI | | |
| Q589R, K591E, | | LALLIAALSHDLDHRGVNNPYIQRSEHPLAQLYCHSI | | |
| S663P, F840S) | | MEHHHFDQCLMILNSPGNQILSGLSIEEYKTTLKIIKQ | | |
| | | AILATDLALYIKRRGEFFELIRKNQFNLEDPHQKELFL | | |
| | | AMLMTACDLSAITKPWPIQQRIAELVATEFFDQGDRE | | |
| | | RKELNIEPTDLMNREKKNKIPSMQVGFIDAICLQLYE | | |
| | | ALTHVSEDCSPLLDGCRKNRQKWQALAEQQ | | |
| 535-860 of | #55 | EETRELQSLAAAVVPSAQTLKITDFSFSDFELSDLETA | 33 | 50 |
| PDE5A (Q589R, | | LCTIRMFTDLNLVQNFRMKHEVLCRWILSVKKNYRK | | |
| I648V, M816T) | | NVAYHNWRHAFNTAQCMFAALKAGKIQNKLTDLEI | | |
| | | LALLVAALSHDLDHRGVNNSYIQRSEHPLAQLYCHSI | | |
| | | MEHHHFDQCLMILNSPGNQILSGLSIEEYKTTLKIIKQ | | |
| | | AILATDLALYIKRRGEFFELIRKNQFNLEDPHQKELFL | | |
| | | AMLMTACDLSAITKPWPIQQRIAELVATEFFDQGDRE | | |
| | | RKELNIEPTDLMNREKKNKIPSTQVGFIDAICLQLYEA | | |
| | | LTHVSEDCFPLLDGCRKNRQKWQALAEQQ | | |
| 535-860 of | #8 | EETRELQSLAAAVVPSAQTLKITDFSLSDLELSDLETA | 34 | 51 |
| PDE5A (F561L, | | LCTIRMFTDLNLVQNFRMKHEVLCRWILSVKKNYRK | | |
| F564L, Q589R, | | NVAYHNWRHAFNTAQCMFAALKAGKIQNKLTDLEI | | |
| D724N, S766F, | | LALLIAALSHDLDHRGVNNSYIQRSEHPLAQLYCHSI | | |
| T802P) | | MEHHHFDQCLMILNSPGNQILSGLSIEEYKTTLKIIKQ | | |
| | | AILATNLALYIKRRGEFFELIRKNQFNLEDPHQKELFL | | |
| | | AMLMTACDLFAITKPWPIQQRIAELVATEFFDQGDRE | | |
| | | RKELNIEPPDLMNREKKNKIPSMQVGFIDAICLQLYE | | |
| | | ALTHVSEDCFPLLDGCRKNRQKWQALAEQQ | | |
| 535-860 of | #25 | EETRELRSLAAAVVPSAQTLKITDLSFSDFELSDLETA | 35 | 52 |
| PDE5A (Q541R, | | LCTIRMFTDLNLVQNFRMKHEVLCRWILSVKKNYRK | | |
| F559L, Q589R, | | NVAYHNWRHAFNTAQCMFAALKAGKIQNKLTDLEI | | |
| H678R, I824T) | | LALLIAAL SHDLDHRGVNNSYIQRSEHPLAQLYCRSI | | |
| | | MEHHHFDQCLMILNSPGNQILSGLSIEEYKTTLKIIKQ | | |
| | | AILATDLALYIKRRGEFFELIRKNQFNLEDPHQKELFL | | |
| | | AMLMTACDLSAITKPWPIQQRIAELVATEFFDQGDRE | | |
| | | RKELNIEPTDLMNREKKNKIPSMQVGFIDATCLQLYE | | |
| | | ALTHVSEDCFPLLDGCRKNRQKWQALAEQQ | | |
| 535-860 of | #3 | EETRELQSLAAAVVPSAQTLKITDFSFSDFELSDLETA | 66 | 70 |
| PDE5A (Q589R, | | LCTIRMFTDLNLVQNFRMKHEVLCRWILSVKKNYRK | | |
| K633E, T712S, | | NVAYHNWRHAFNTAQCMFAALKAGEIQNKLTDLEI | | |
| K852E, K795E) | | LALLIAALSHDLDHRGVNNSYIQRSEHPLAQLYCHSI | | |
| | | MEHHHFDQCLMILNSPGNQILSGLSIEEYKTSLKIIKQ | | |
| | | AILATDLALYIKRRGEFFELIRKNQFNLEDPHQKELFL | | |
| | | AMLMTACDLSAITKPWPIQQRIAELVATEFFDQGDRE | | |
| | | REELNIEPTDLMNREKKNKIPSMQVGFIDAICLQLYE | | |
| | | ALTHVSEDCFPLLDGCRKNRQEWQALAEQQ | | |
| 535-860 of | #53 | EGTRELQSLAAAVVPSAQTLKITDFSFSDFELSDLETA | 67 | 71 |
| PDE5A (E536G, | | LCTIRMFTDLNLVQNFRMKHEVLCRWILSVKKNYRK | | |
| Q589R, C839S) | | NVAYHNWRHAFNTAQCMFAALKAGKIQNKLTDLEI | | |
| | | LALLIAALSHDLDHRGVNNSYIQRSEHPLAQLYCHSI | | |
| | | MEHHHFDQCLMILNSPGNQILSGLSIEEYKTTLKIIKQ | | |
| | | AILATDLALYIKRRGEFFELIRKNQFNLEDPHQKELFL | | |
| | | AMLMTACDLSAITKPWPIQQRIAELVATEFFDQGDRE | | |
| | | RKELNIEPTDLMNREKKNKIPSMQVGFIDAICLQLYE | | |
| | | ALTHVSEDSFPLLDGCRKNRQKWQALAEQQ | | |
| 535-860 of | #57 | EETRELQSLAAAVVPSAQTLKITDFSFSDFELSDLETA | 68 | 72 |
| PDE5A (N587S, | | LCTIRMFTDLNLVQSFRMKHEVLCRWILSVKKNYRE | | |
| Q589R, K608E, | | NVAYHNWRHAFNTAQCMFAALKAGKIQNKLTDLEI | | |
| N661S, D764V) | | LALLIAALSHDLDHRGVSNSYIQRSEHPLAQLYCHSI | | |
| | | MEHHHFDQCLMILNSPGNQILSGLSIEEYKTTLKIIKQ | | |
| | | AILATDLALYIKRRGEFFELIRKNQFNLEDPHQKELFL | | |
| | | AMLMTACVLSAITKPWPIQQRIAELVATEFFDQGDRE | | |
| | | RKELNIEPTDLMNREKKNKIPSMQVGFIDAICLQLYE | | |
| | | ALTHVSEDCFPLLDGCRKNRQKWQALAEQQ | | |
| 535-860 of | #30 | EETRELQSLAAAVVPSAQTLKITDFSFSDFELSDLETA | 69 | 73 |
| PDE5A (Q589R, | | LCTIRMFTDLNLVQNFRMKHEVLCRWILSVKKNYRK | | |
| L675P, F755L) | | NVAYHNWRHAFNTAQCMFAALKAGKIQNKLTDLEI | | |
| | | LALLIAALSHDLDHRGVNNSYIQRSEHPLAQPYCHSI | | |
| | | MEHHHFDQCLMILNSPGNQILSGLSIEEYKTTLKIIKQ | | |
| | | AILATDLALYIKRRGEFFELIRKNQFNLEDPHQKELLL | | |
| | | AMLMTACDLSAITKPWPIQQRIAELVATEFFDQGDRE | | |
| | | RKELNIEPTDLMNREKKNKIPSMQVGFIDAICLQLYE | | |
| | | ALTHVSEDCFPLLDGCRKNRQKWQALAEQQ | | |

In some embodiments, the DDs derived from PDE5A may comprise one, two, three, four, five or more mutations to the catalytic domain of PDE5A selected from E5351D, E536G, Q541R, K555R, F559L, S560G, F561L, F564L, F564S, V585A, N587S, K591E, 1599V, K604E, K608E, N609H, K630R, K633E, N636S, 1648V, N661S, S663P, L675P, S766F, Y676D), Y676N, C677R, H678R, D687A, T711A, T712S, D724N, L738H, N742S, F744L, L746S, F755L, A762S, D764V, D764N, D764G, K795E, L797F, 1799T, L804P, T802P, S815C, M816A, M816T, I824T, C839S, F840S, and K852E.

The amino acid sequences of the destabilizing domains, in some embodiments, have at least about 40%, 50 or 60% identity, further at least about 70% identity, preferably at least about 75% or 80% identity, more preferably at least about 85%, 86%, 87%, 88%, 89% or 90% identity, and further preferably at least about 91%, 92%, 93%, 94%, 95%, 96%, 97%, 98% or 99% identity to the amino acid sequence set forth therein. Percent identity may be determined, for example, by comparing sequence information using the advanced BLAST computer program, including version Magic-BLAST 1.2.0, available from the National Institutes of Health. The BLAST program is based on the alignment method discussed in Karl and Altschul (1990) Proc. Nadl. Acad. Sci USA, 87:2264-68 (the contents of which are incorporated by reference in their entirety). Accordingly, PDE5A sequence of SEQ ID NO. 1 was analyzed using BLAST and sequences that demonstrated up to 40% homology were identified. This analysis identified Phosphodiesterase 11 isoform 1, Phosphodiesterase 11 isoform 2, Phosphodiesterase 11 isoform 3 and Phosphodiesterase 11 isoform 4 which bear approximately 40% homology to PDE5A. DDs may in some instances be derived from PDE11 isoforms.

In some embodiments, PDE5A derived destabilizing domains may be identified by the method of walk-through mutagenesis. The method of walk through mutagenesis consists of introducing a predetermined amino acid in to each position in a predefined region (or several different regions) of the amino acid sequence of a parent polypeptide. A PDE5A protein library is generated which contains multiple mutant PDE5A proteins, each having the predetermined amino acid in one or more positions in the region and collectively, in every position in the region. In some embodiments, predetermined regions selected for mutagenesis may include regions of PDE5A which are enriched for destabilizing mutations. In some embodiments, the predetermined region for mutagenesis may be selected from a portion of PDE5A of SEQ ID NO. 1, wherein the predetermined region spans from 531-541, position 555-564, position 587-591, position 604-609, position 630-636, position 674-678, position 762-766, position 795-797; a position 809-816 or any combination of thereof. This method allows for systematic evaluation of the role of a specific amino acid in the ligand dependent stabilization. Walk through mutagenesis methods are described in further detail in U.S. Pat. Nos., U.S. Pat. Nos. 6,649,340, 5,830,650, and 5,798,208 (the contents of which are incorporated by reference in their entirety).

Amino acid substitutions utilized for methods described herein may be conservative or non-conservative substitutions. Conservative amino acid substitutions may be made in the amino acid sequences described herein to obtain derivatives of the peptides that may advantageously be utilized. Conservative amino acid substitutions, as known in the art and as referred to herein, involve substituting amino acids in a protein with amino acids having similar side chains in terms of, for example, structure, size and/or chemical properties. For example, the amino acids within each of the following groups may be interchanged with other amino acids in the same group: amino acids having aliphatic side chains, including glycine, alanine, valine, leucine and isoleucine; amino acids having acidic side chains, such as aspartic acid and glutamic acid; amino acids having acidic side chains, including glutamine and asparagine; basic amino acids, including lysine, arginine and histidine; amino acids having aromatic ring side chains, including phenylalanine, tyrosine, and tryptophan; and amino acids having sulfur containing side chains, including cysteine and methionine. Additionally, amino acids having acidic side chains, such as aspartic acid and glutamic acid, can often be substituted with amino acids having amide side chains such as asparagine and glutamine.

The destabilizing domains may also be fragments of the above described destabilizing domains, including fragments containing variant amino acid sequences. Preferred fragments are unstable in the absence of a stabilizing ligand and stabilized by the presence of ligand. Such fragments are readily identified using assays described herein. Preferred fragments retain the ability to bind to a stabilizing ligand with similar efficiency to the destabilizing domains described herein or with at least 90% efficiency, or at least 80% efficiency, at least 70% or at least 50% efficiency with respect to the described destabilizing domains. Preferred destabilizing domains may be fused at the N terminus or C terminus of a POI.

### Ligands

Stabilizing ligands for use include Sildenafil (i.e. 5-[2-ethoxy-5-(4-methylpiperazin-1-yl) sulfonylphenyl]-1-methyl-3-propyl-4H-pyrazolo[4,3-d]pyrimidin-7-one), a commercially available PDE5A inhibitor. In some embodiments, ligands known to bind to full length or a portion of PDE5A may be utilized as stabilizing ligands. For example, stabilizing ligands may include but are not limited to known PDE5A inhibitors, including Vardenafil, Tadalafil, Avanafil, Lodenafil, Mirodenafil, Udenafil, Benzamidenafil, Dasantafil, and Beminafil. Other stabilizing ligands that may be useful include Sildenafil-derived ligands containing portions of the ligand known to mediate binding to PDE5A. Ligands may also be modified to reduce off-target binding to Phosphodiesterases and increase specific binding to PDE5A. Stabilizing ligands may also be selected through the analysis of the dependence of the stabilizing capability of the ligand on its chemical structure, through Structure Activity Relationships (SAR) studies. Any of the methods related to SAR, known in art may be utilized to identify stabilizing ligands.

### Proteins of Interest

In some embodiments, proteins of interest may be a natural protein in an organism genome, or variants, mutants, derivatives thereof. The natural protein may be from, for example, a mammalian organism, a bacterium, and a virus. In one example, the protein of interest, or a polypeptide may be derived from human genome.

In some embodiments, the proteins of interest may be selected from, but are not limited to enzymes, structural proteins, signaling proteins, regulatory proteins, transport or carrier proteins, sensory proteins, motor proteins, immune proteins, and storage proteins.

Proteins of interest may also be selected from any known reporter protein, which refers to any protein capable of creating a detectable signal, in response to an input. Examples include alkaline phosphatase, β-galactosidase, chloramphenicol acetyltransferase, β-glucuronidase, peroxidase, β-lactamase, catalytic antibodies, bioluminescent proteins e.g. luciferase, and fluorescent proteins such as Green fluorescent protein (GFP).

### III. Methods, Cells and Transduced Animals

Also disclosed is method for conditionally stabilizing a protein of interest, which consists of fusing a nucleic acid encoding the protein of interest fused in-frame to a nucleic acid encoding a destabilizing domain, optionally in combination with one or more of the substitutions identified herein. In one example, the cells are transfected with nucleic acids encoding a fusion protein comprising a protein of interest fused in frame to a variant PDE5A protein. Expression of the DD fusion protein may be driven by the endogenous promoter, ideally reproducing the spatial and temporal expression patterns of the unmodified gene.

The cells may be transfected, e.g. using an expression vector or transduced (i.e. infected) using a viral vector, including, but not limited to a vector derived from a retrovirus (e.g. a lentivirus), herpesvirus, pox virus, adenovirus, adeno-associated virus, or an RNA virus, such as poliovirus, flavivirus, alphavirus or the like. The exemplary viral vector is a retrovirus.

The cells may be eukaryotic cells, including but not limited to cells from humans, primates, rodents, and other animals, including domesticated animals. The cells may also be from plants, insects, amphibians, and apicomplexan parasites. The cells may be in culture or in a living organism. The wild-type or naturally occurring gene or allele encoding the POI may be deleted to facilitate study of the conditionally stabilized POI.

The present methods and compositions also allow the creation of transgenic animals harboring engineered alleles that direct the expression of a ligand stabilized POI. Expression of the DD fusion protein may be driven by the endogenous promoter ideally reproducing the spatial and temporal expression patterns of the unmodified gene. The ligand may be administered regularly from an early age (including in utero) to stabilize the fusion protein until the mice achieve a specified age, at which time, withdrawal of the ligand results in a rapid degradation of the fusion protein. Unlike Cre-mediated gene disruption, this method is reversible, such that simply reinitiating the administration of the ligand, allows the rapid, reversible and conditional control of protein function in a complex system.

The ability to specifically and conditionally stabilize a POI in a cell will enable the study of many proteins to determine their biological function and importance in a cell. The present methods and compositions represent a significant improvement over current methods of conditional protein regulation.

### IV. Definitions

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, mean that the moieties are physically associated or connected with one another, either directly or via one or more additional moieties that serve as linking agents, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Conservative amino acid substitutions: As used herein, a “conservative amino acid substitutions” are substitutions that do not result in a significant change in the activity or tertiary structure of a selected polypeptide or protein. Such substitutions typically involve replacing a selected amino acid residue with a different residue having similar physico-chemical properties. For example, substitution of glutamine for aspartate is considered a conservative substitution since both are similarly-sized negatively-charged amino acids. Groupings of amino acids by physico-chemical properties are known to those of skill in the art.

Domain: As used herein, the terms “domain” and “region” are used interchangeably herein and refer to a contiguous sequence of amino acids within a POI or destabilizing domain, typically characterized by being either conserved or variable and having a defined function, such as ligand binding, conferring stability or instability, enzymatic function, etc.

Degradation: As used herein, “degradation” or “destruction” of a protein means its hydrolysis into smaller proteins or amino acids, such as by the cellular proteasome.

Destabilized: As used herein, the term “destable,” “destabilize,” “destabilizing region” or “destabilizing domain” means a region or molecule that is less stable than a starting, reference, wild-type or native form of the same region or molecule.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; (4) folding of a polypeptide or protein; and (5) post-translational modification of a polypeptide or protein.

Fused: As used herein, “fused” means arranged in-frame as part of the same contiguous sequence of amino acids in a polypeptide. Fusion can be direct such that there are no additional amino acid residues or via a linker to improve performance or add functionality.

Homologous: Two amino acid sequences or two nucleotide sequences are considered “homologous” if they have an alignment score of >5 (in standard deviation units) using the program ALIGN with the mutation gap matrix and a gap penalty of 6 or greater (Dayhoff, M. O., in Atlas of Protein Sequence and Structure (1972) Vol. 5, National Biomedical Research Foundation, pp. 101-110, and Supplement 2 to this volume, pp. 1-10.) The two sequences (or parts thereof) are more preferably homologous if their amino acids are greater than or equal to 50%, 70%, 80%, 90%, 95%, or even 98% identical when optimally aligned using the ALIGN program mentioned above.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Introduction of nucleic to cells: As used herein, “introduction of nucleic to cells” means transfection, transduction (infection), or transformation of nucleic acids (e.g., DNA) into cells, such that the nucleic acids may be used by the cell to express a protein of interest.

Modulate: “Modulate” intends a lessening, an increase, or some other measurable change, e.g., in the stability or biological function of a protein.

Mutant: As used herein, a “mutant” is a mutated protein designed or engineered to alter properties or functions relating to protein stabilization and/or ligand binding.

Protein of interest (POI): As used herein, a “protein of interest” or “POI” is any protein, or functional fragment or derivative, thereof, that one skilled in the art wishes to study.

Preferentially binds: As used herein, “preferentially binds” means to bind with greater efficiency to a subject molecule (such as a particular amino acid sequence) than another molecule. The difference in binding efficiency may be 5-fold, 10-fold, 50-fold, 100-fold, 500-fold, 1,000-fold, 10,000-fold, or more.

PDE5A variant: “PDE5A variant” refers to a protein wherein one or more amino acid residues, are substituted for an amino acid other than the amino acid in the PDE5A (SEQ ID NO. 1). Other amino acid positions that can be substituted are indicated in the Tables and Figures.

Protein: As used herein, the terms “protein” and “polypeptide” are used interchangeably and without distinction to refer to a compound made up of a chain of amino acid residues linked by peptide bonds. Unless otherwise indicated, the sequence for peptides is given in the order from the “N” (or amino) terminus to the “C” (or carboxyl) terminus. It is understood that polypeptides include a contiguous sequence of amino acid residues.

Peptide: A peptide or peptide fragment is “derived from” a parent peptide or polypeptide if it has an amino acid sequence that is homologous to, but not identical to, the parent peptide or polypeptide, or of a conserved fragment from the parent peptide or polypeptide.

Small molecule ligand: A “small molecule ligand” is a discrete small-molecule, well known in the pharmaceutical and material sciences, which is to be distinguished from, e.g., a polypeptide or nucleic acids, which is a polymer consisting of monomeric subunits. Small molecule ligands may be naturally-occurring or synthetic as exemplified by pharmaceutical products, laboratory reagents, and the like.

Stable: As used herein “stable” refers to a compound or entity that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable. In some embodiments, stability is measured relative to an absolute value. In some embodiments, stability is measured relative to a secondary status or state or to a reference compound or entity.

Variant: As used herein, a “variant” protein is a protein having an amino acid sequence that does not occur in nature, as exemplified by sequences in GenBank.

### V. Kits of Parts

The methods and compositions described herein may be packaged together with instructions for use, as in a kit of parts. Preferred kits of parts include a PDE5A variant, a stabilizing ligand, and instructions for use. The PDE5A variant may include a substitution described herein, or may include other substitutions identified using the methods decried herein. The stabilizing ligand may be as described herein or a variant or derivative that functions in a similar manner. Instructions may include the nucleotide sequences of a plasmid encoding a PDE5A variant and instructions and guidelines for inserting (i.e. cloning) a POI into the plasmid—in frame with the PDE5A variant. The instructions may also include dosing recommendations and hardware such as syringes, to deliver the fusion protein to an organism or to cells.

### Examples

### Example 1. PDE5A Library Generation

Diversity in the PDE5A catalytic domain sequence (Q589R) (SEQ ID NO. 11) was generated using a combination of error-prone PCR and nucleotide analog mutagenesis. Primers for mutagenic PCR were designed to anneal upstream of the 5′ restriction site to be used for cloning the mutagenesis products into pBMN iHcRed—tandem retroviral expression vector and to anneal downstream of the 3′ restriction site. Three independent conditions were used to generate diversity. Condition set A utilized 4 ng template, 0.5 μM of each oligonucleotide primer, 5 units Taq polymerase, 5 mM MgCl₂, 0.2 mM MnCl₂, 0.4 mM dNTPs in equal ratio, and an excess of 0.2 mM dATP and dCTP. Condition set B was identical to A, except that dGTP and dTTP were present in excess. Condition set C utilized the non-natural nucleotides 8-oxo-dGTP and dPTP to encourage nucleotide mis-incorporation. The PDE5A libraries were pooled and ligated into the pBMN iHcRed-t retroviral expression vector, affording a library containing approximately 3×10⁴ members.

### Example 2. Cell Culture and Transduction

The NIH3T3 cell line was cultured in DMEM supplemented with 10% heat-inactivated donor bovine serum (Invitrogen), 2 mM glutamine, 100 U/ml penicillin and 100 μg/ml streptomycin.

The ΦNX ecotropic packaging cell line was transfected using standard Lipofectamine 2000 protocols. Viral supernatants were harvested 48 hours, post transfection, filtered and concentrated 10-fold using Amicon Ultra centrifugal filter device with a 100 kDa cutoff. NIH 3T3 cells were incubated with concentrated retroviral supernatants supplemented with 4 μg/ml polybrene for 4 hours at 37° C. Cells were washed once with PBS and cultured in growth media for 24-36 hours to allow for viral integration.

### Example 3. Fluorescence-Activated Cell Sorting (FACS)

The transduced NIH3T3 cells were subjected to analysis using fluorescence-activated cell sorting (FACS) to screen the libraries of PDE5A mutants. 24 hours prior to analysis, transduced NIH3T3 cells were plated at 1×10⁵ cells per well of a 12-well plate. Cells were removed from the plate using PBS and 2 mM EDTA, washed once with PBS, and resuspended in 200 μL PBS. FACS analysis was performed on instruments at the Stanford Shared FACS Facility with 10,000 events represented.

### Example 4. Cell-Based Screening Assay

To identify destabilizing domains that are responsive to the PDE5A ligand, Sildenafil, a cell-based screening assay was employed in which the fluorescence of green fluorescent protein derived from Aequorea coerulescens (AcGFP) served as an indicator of PDE5A stability. A library based on the PDE5A catalytic domain sequences was generated using error prone PCR and then cloned in-frame in front of AcGFP. A Moloney murine leukemia retroviral expression was used to stably integrate this library of PDE5A-AcGFP fusions into NIH-3T3 fibroblasts, and the transduced cells were subjected to three rounds of sorting using flow cytometry. In the first round, cells were treated with 10 μM of PDE5A ligand, Sildenafil for 16-24 before analysis. Fluorescent cells were collected and further cultured in the absence of ligand for 60 hours. Analysis revealed that approximately 5% of the cell population exhibited decreased fluorescence levels indicating that most the sequences were either unmutated or contained mutations that did affect the stability of the fusion protein. This small population of cells exhibiting decreased fluorescence was collected and cultured once more in the presence of 10 μM Sildenafil for 24 hours, at which time AcGFP-expressing cells were collected and the genomic DNA was isolated.

### Example 5. Identification of C-Terminal PDE5A-Derived Destabilizing Domains

A library of AcGFP-PDE5A fusions i.e., C-terminal PDE5A mutants was screened using the cell-based screening assay described previously to identify C-terminal destabilizing domains. Several clones that exhibited desired characteristics, i.e., low basal expression in the absence of ligand and high expression in the presence of ligand were selected that. Identified candidates were subjected to further characterization by FACS. Cells were split into two treatment groups and incubated with either Sildenafil (10 μM) or vehicle alone for 24 hours. Cells that were mock transduced or transduced with the wild-type PDE5A construct were also included in the analysis. The stabilization ratio was calculated as the fold change in GFP intensity of the AcGFP-PDE5A construct in Sildenafil treated sample compared to vehicle alone treated sample. The results are presented in Table 2 where the C terminal PDE5A mutants are denoted as PDE5AC. Among the identified mutants, PDE5AC (#2) and PDE5AC (#41) showed stabilization ratios greater than 6 suggesting strong ligand dependent stabilization.

TABLE 2
FACS analysis of C-terminal PDE5A mutants

| | | Mean | |
|---|---|---|---|
| | Sildenafil | Fluorescence | Stabilization |
| Clone No. | (10 μM) | intensity | ratio |
| NIH3T3 control | − | 345 | — |
| PDE5AC | − | 9520 | 0.9 |
| Wild-type | + | 8797 | |
| PDE5AC (#2) | − | 1023 | 6.5 |
| | + | 6577 | |
| PDE5AC (#8) | − | 1066 | 3.1 |
| | + | 3310 | |
| PDE5AC (#13) | − | 1076 | 5.8 |
| | + | 6284 | |
| PDE5AC (#25) | − | 1452 | 1.6 |
| | + | 2340 | |
| PDE5AC (#28) | − | 1382 | 4.4 |
| | + | 6055 | |
| PDE5AC (#40) | − | 1255 | 4.7 |
| | + | 5923 | |
| PDE5AC (#41) | − | 1000 | 6.1 |
| | + | 6115 | |
| PDE5AC (#42) | − | 1658 | 3.5 |
| | + | 5861 | |
| PDE5AC (#43) | − | 1017 | 5.4 |
| | + | 5518 | |
| PDE5AC (#69) | − | 1126 | 5.6 |
| | + | 6365 | |

Identified PDE5A mutants were further validated using immunoblot analysis. NIH3T3 cells expressing AcGFP-PDE5A fusions were incubated with 10 μM Sildenafil or vehicle alone for 24 hours and the stability of PDE5A mutants was evaluated at the protein level. Cell lysates obtained from transduced NIH3T3 cells were immunoblotted using the anti-XFP antibody (1:3000) at 4° C. overnight. Samples were also immunoblotted with anti-GAPDH antibody (1:10000) to ensure uniform protein loading. Samples were then incubated with secondary anti-mouse antibody (1:3000) at room temperature for 1 hour, followed by visualization using appropriate methods. Immunoblot results are presented in FIG. 2. All PDE5A mutants demonstrated enhanced GFP expression in the presence of the ligand. The difference in band intensity recapitulates the pattern from the FACS analysis. Consistent with the FACS analysis, PDE5AC (#2) and PDE5AC (#41) displayed most drastic difference in the intensity of the bands from mock treated sample and Sildenafil treated sample, indicating strong ligand dependent stabilizing effect.

Identified PDE5A mutants were sequenced and sequences were compared to the wild-type sequence of human PDE5A catalytic domain to identify mutations. The mutations identified are shown in Table 3 where the C terminal PDE5A mutants are denoted as PDE5AC. The number of amino acid mutations was calculated based on the wildtype PDE5A amino acid sequence of SEQ ID NO. 1.

TABLE 3
Sequence analysis of C-terminal PDE5A mutants

| | No. of | | |
|---|---|---|---|
| | amino | | Protein |
| | acid | | SEQ ID |
| Clone No. | mutations | Amino acid mutations | NO. |
| PDE5AC (#2) | 4 | Q589R, D687A, D764N, S815C | 23 |
| PDE5AC (#13) | 3 | Q589R, Y676D, L738H | 24 |
| PDE5AC (#28) | 4 | Q589R, K591E, N609H, D764V | 25 |
| PDE5AC (#40) | 2 | Q589R, D764G | 26 |
| PDE5AC (#41) | 5 | F561L, Q589R, K604E, | 27 |
| | | D724N, L797F | |
| PDE5AC (#42) | 9 | E535D, K555R, F564S, Q589R, | 28 |
| | | K630R, C677R, N742S, | |
| | | I799T, M816A | |
| PDE5AC (#43) | 4 | Q589R, N609H, Y676N, A762S | 29 |
| PDE5AC (#69) | 5 | Q589R, N636S, D687A, | 30 |
| | | D764N, S815C | |
| PDE5AC (#8) | 5 | F561L, F564L, Q589R, | 34 |
| | | D724N, S766F, T802P | |
| PDE5AC (#25) | 5 | Q541R, F559L, Q589R, | 35 |
| | | H678R, I824T | |

Sequence analysis (see Table 3) revealed that residue D764 as a frequently mutated residue. D764G alone in Clone 40 induced 4.7-fold of ligand dependent stabilization, suggesting that it may be important for PDE5A protein stability. The error prone mutagenesis also introduced several silent mutations which was evident by the analysis of the nucleotide sequence.

### Example 6. Optimization of C-Terminal PDE5A Mutants

Identified C-terminal PDE5A mutants were further optimized to improve ligand dependent stabilization properties. Additional point mutations or linker sequences were evaluated for their effect on PDE5A mutant characteristics.

### D764 Mutation

Residue D764 was identified as a mutational hotspot from sequence analysis. PDE5AC (#41) was engineered with either D764N or D764G mutation via site-directed mutagenesis. PDE5AC (#41)+D764N comprises the amino acid sequence of SEQ ID NO. 53 (encoded by SEQ ID NO. 54) and PDE5AC (#41)+D764G comprises the amino acid sequence of SEQ ID NO. 55 (encoded by SEQ ID NO. 56). AcGFP-PDE5A variants were evaluated for Sildenafil dependent stabilization via FACS as described previously.

Results from FACS analysis are presented in Table 4 where the C terminal PDE5A mutants are denoted as PDE5AC. The stabilization ratio was calculated as the fold change in GFP intensity of the AcGFP-PDE5A construct in Sildenafil treated sample compared to vehicle alone treated sample.

TABLE 4
Effect of D764 mutations

| | | Mean | |
|---|---|---|---|
| | Sildenafil | Fluorescence | Stabilization |
| Clone No. | (10 μM) | intensity | ratio |
| NIH3T3 control | − | 303 | — |
| Wild-type | − | 6992 | 1.0 |
| | + | 7093 | |
| PDE5AC (#2) | − | 671 | 5.2 |
| | + | 3483 | |
| PDE5AC (#40) | − | 917 | 4.6 |
| | + | 4229 | |
| PDE5AC (#41) | − | 734 | 6.3 |
| | + | 4647 | |
| PDE5AC (#41) + | − | 709 | 1.0 |
| D764G | + | 730 | |
| PDE5AC (#41) + | − | 684 | 1.5 |
| D764N | + | 1007 | |

The results in Table 4, show that addition of D764G or D764N to PDE5AC (#41) did not improve its ligand dependent stabilization but rather abolished its responsiveness to Sildenafil as indicated by a reduction in the stabilization ration upon addition of ligand. These data show that effect of the mutations at the D764 position is dependent on the context of other mutations present on PDE5A. The combination of mutations in PDE5AC (#41), requires further optimization.

### Protein Tags

A hemagglutinin (HA) sequence (SEQ ID NO. 74; encoded by SEQ ID NO. 75) was cloned in-frame at either the 5′- or 3′-ends of the PDE5A mutant sequence from PDE5AC (#41). The construct further included AcGFP (amino acids 1-239 of WT) at the N terminus (SEQ ID NO. 76, encoded by SEQ ID NO. 77) and an EF linker/EcoRI restriction site (GAATTC). The resulting fusion protein adopts either the AcGFP (1-239 of WT)-EF linker-HA-PDE5AC-stop (#41) configuration with an amino acid sequence of SEQ ID NO. 57 (encoded by SEQ ID NO. 58) or AcGFP-EF-Linker-PDE5AC (#41)-HA-stop configuration with an amino acid sequence of SEQ ID NO. 59 (encoded by SEQ ID NO. 60). The effect of the HA sequence on ligand dependent stabilization was evaluated via immunoblot analysis. The transduced NIH3T3 cells were incubated with 10 μM Sildenafil or vehicle alone for 24 hours. Cell lysates obtained from transduced NIH3T3 cells were immunoblotted using either the anti-XFP antibody (1:3000) or anti-HA antibody (1:2000). As show in FIG. 3, HA-tagged fusion protein expression detected with either anti-XFP antibody or anti-HA antibody was nearly identical. AcGFP-HA-PDE5A (#41) exhibited similar level of ligand dependent stabilization compared to Clone 41. However, no protein expression was detected from AcGFP-PDE5A (#41)—HA in either Sildenafil treated sample or mock treated sample. Additional protein tag sequences may be explored to be used in conjunction with destabilizing domains.

### Example 7. Evaluation of Extended PDE5A Variants

Extended versions of human PDE5A that included residues in addition to catalytic domain (residues 535-860 of SEQ ID NO. 1) with Q589R polymorphism (SEQ ID NO. 11) were evaluated for use as C-terminal destabilizing domains. Three extended PDE5A sequences were tested that consisted of residues 535-875 of SEQ ID NO. 1 with Q589R polymorphism (SEQ ID NO. 13); residues 466-875 of SEQ ID NO. 1 with Q589R polymorphism (SEQ ID NO. 15); or residues 420-875 of SEQ ID NO. 1 with Q589R polymorphism (SEQ ID NO. 17). These PDE5A sequences were cloned to the 3′ end of AcGFP and the AcGFP fusions were assessed for ligand dependent stabilization using FACS as described previously. Results of FACS analysis are presented in Table 5 where the C terminal PDE5A mutants are denoted as PDE5AC. The stabilization ratio was calculated as the fold change in GFP intensity of the AcGFP-PDE5A construct in Sildenafil treated sample compared to vehicle alone treated sample.

TABLE 5
FACS analysis of extended PDE5A variants

| | | Mean | |
|---|---|---|---|
| | Sildenafil | Fluorescence | Stabilization |
| Construct | (10 μM) | intensity | ratio |
| NIH3T3 control | − | 409 | — |
| PDE5AC 535-860 | − | 8431 | 1.0 |
| (Q589R) | + | 8557 | |
| PDE5AC 535-875 | − | 6761 | 1.0 |
| (Q589R) | + | 6462 | |
| PDE5AC 466-875 | − | 819 | 1.1 |
| (Q589R) | + | 888 | |
| PDE5AC 420-875 | − | 620 | 0.9 |
| (Q589R) | + | 572 | |

The extended PDE5A variants displayed reduced stability in the absence of Sildenafil compared to the PDE5A catalytic domain (535-860) (Q589R). The reduction was most substantial in PDE5AC 466-875 (Q589R) and PDE5AC 420-875 (Q589R). However, none of the extended PDE5A variants showed ligand responsive stabilization in the presence of Sildenafil. Further, PDE5AC (466-875) (Q589R) and PDE5AC (420-875) (Q589R) showed low basal expression in the absence of ligand while PDE5AC (535-860) (Q589R) and PDE5AC (535-875) (Q589R) showed high basal expression in the absence of ligand. These data show that PDE5AC (535-860) (Q589R) and PDE5AC (535-875) (Q589R) may be suitable for screening and identification of mutations that destabilize PDE5AC since they have high basal expression. This analysis may be utilized in combination with the mutagenesis analysis to identify additional destabilizing domains.

### Example 8. Sildenafil Dose Dependency

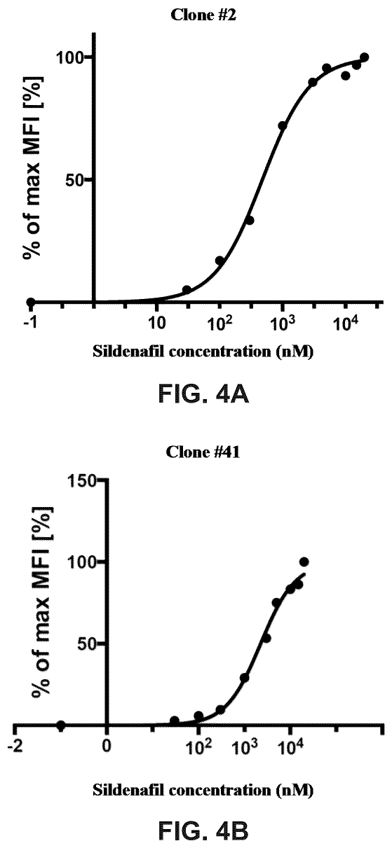

NIH3T3 cells expressing AcGFP-PDE5A variants were incubated with varying concentrations of Sildenafil ranging from 0.003 μM to 30 μM. The stability of PDE5A mutants was measured using FACS and mean fluorescence intensity (MFI) of GFP was calculated. Maximum MFI was observed at highest Sildenafil concentration. Percentage of max MFI (%) was calculated as the ratio of the MFI at a ligand concentration compared to the maximum MFI. Percentage of max MFI was plotted against the concentration of Sildenafil presented in a log scale. Titration curves of PDE5AC (#2) and PDE5AC (#41) are presented in FIG. 4A and FIG. 4B, respectively. Both clones showed an increase in percentage of max MFI with increasing doses of Sildenafil, indicating a ligand dose-dependent stabilization of PDE5A mutants. The half maximal effective concentration or EC₅₀ was approximately 0.5 μM for PDE5AC (#2) and 2 μM for PDE5AC (#41).

### Example 9. Identification of N-Terminal PDE5A-Derived Destabilizing Domains

A library of PDE5A-AcGFP fusions i.e., N-terminal PDE5A mutants, was screened using the cell-based screening assay described previously to identify N-terminal destabilizing domains. Several clones were selected that exhibited desired characteristics, i.e., low basal expression in the absence of ligand and high expression in the presence of ligand. Identified candidates were subjected to further characterization by FACS. Cells were split into two treatment groups and incubated with either Sildenafil (20 μM) or vehicle alone for 24 hours. Cells that were mock transduced or transduced with the wild-type PDE5A construct were also included in the analysis. The results are presented in Table 6 where the N terminal PDE5A mutants are denoted as PDE5AN. The stabilization ratio was calculated as the fold change in GFP intensity of the PDE5A-AcGFP construct in Sildenafil treated sample compared to vehicle alone treated sample.

TABLE 6
FACS analysis of N-terminal PDE5A mutants

| | Mean Fluorescence | | |
|---|---|---|---|
| | Intensity (MFI) | | |
| | Vehicle | Sildenafil | Stabilization |
| Construct | control | (20 μM) | Ratio |
| 3T3 | 234 | — | — |
| PDE5AN (#51) | 1603 | 1543 | 0.96 |
| PDE5AN (#64) | 991 | 966 | 0.97 |
| PDE5AN (#52) | 224 | 219 | 0.98 |
| PDE5AN (#36) | 230 | 230 | 1.00 |
| PDE5AN (#56) | 1052 | 1054 | 1.00 |
| PDE5AN (#4) | 213 | 218 | 1.02 |
| PDE5AN (#46) | 217 | 227 | 1.05 |
| PDE5AN (#28) | 468 | 501 | 1.07 |
| PDE5AN (#61) | 219 | 235 | 1.07 |
| PDE5AN (#42) | 223 | 240 | 1.08 |
| PDE5AN (#62) | 210 | 227 | 1.08 |
| PDE5AN (#17) | 204 | 224 | 1.10 |
| PDE5AN (#10) | 1157 | 1279 | 1.11 |
| PDE5AN (#14) | 206 | 228 | 1.11 |
| PDE5AN (#32) | 933 | 1074 | 1.15 |
| PDE5AN (#43) | 3407 | 4372 | 1.28 |
| PDE5AN (#18) | 3199 | 4523 | 1.41 |
| PDE5AN (#24) | 1603 | 2290 | 1.43 |
| PDE5AN (#59) | 535 | 1051 | 1.96 |
| PDE5AN (#41) | 920 | 1877 | 2.04 |
| PDE5AN (#9) | 1007 | 2215 | 2.20 |
| PDE5AN (#50) | 960 | 2113 | 2.20 |
| PDE5AN (#1) | 1488 | 3508 | 2.36 |
| PDE5AN (#35) | 1476 | 3684 | 2.50 |
| PDE5AN (#11) | 1143 | 2875 | 2.52 |
| PDE5AN (#53) | 1697 | 4440 | 2.62 |
| PDE5AN (#19) | 1502 | 4297 | 2.86 |
| PDE5AN (#55) | 1257 | 3602 | 2.87 |
| PDE5AN (#26) | 712 | 2633 | 3.70 |
| PDE5AN (#3) | 839 | 3335 | 3.97 |
| PDE5AN (#30) | 1056 | 4713 | 4.46 |
| PDE5AN (#57) | 770 | 5851 | 7.60 |

Among the analyzed mutants, PDE5AN (#57) exhibited highest stabilization ration of 7.6 indicating a strong stabilization as a result of low GFP intensity in the absence of ligand and high GFP intensity in the presence of ligand. A stabilization ratio greater than 1 was observed with all constructs except PDE5AN (#51), PDE5AN (#64), PDE5AN (#52), PDE5AN (#36), and PDE5AN (#56) indicating some level of ligand induced stabilization. Select PDE5A mutants were sequenced and sequences were compared to the wild-type sequence of human PDE5A catalytic domain to identify mutations. Sequence analysis (see Table 7) reveals point mutations different from those in the C-terminal mutants. Interestingly, a D764V mutation also was identified in PDE5AN (#57), highlighting its role in PDE5A structural stability. The number of amino acid mutations in Table 7 was calculated based on the wildtype PDE5A amino acid

TABLE 7
Sequence analysis of N-terminal PDE5A mutants

| | No. of | | |
|---|---|---|---|
| | amino | | Protein |
| | acid | | SEQ ID |
| Clone No. | mutations | Amino acid mutations | NO. |
| PDE5AN (#3) | 5 | Q589R, K633E, T712S, | 66 |
| | | K852E, K795E | |
| PDE5AN (#53) | 3 | E536G, Q589R, C839S | 67 |
| PDE5AN (#57) | 5 | N587S, Q589R, K608E, | 68 |
| | | N661S, D764V | |
| PDE5AN (#30) | 3 | Q589R, L675P, F755L | 69 |
| PDE5AN (#19) | 6 | Q589R, I599V, T711A, | 31 |
| | | F744L, L746S, L804P | |
| PDE5AN (#35) | 7 | S560G, V585A, N587S, | 32 |
| | | Q589R, K591E, S663P, | |
| | | F840S | |
| PDE5AN (#55) | 3 | Q589R, I648V, M816T | 33 |

### Example 10. Comparison of N-Terminal and C-Terminal PDE5A-Derived Destabilizing Domains

The ability of PDE5A derived DDs to destabilize a protein of interest may depend on the position whether the DD is appended to the N terminus or the C terminus of the protein of interest. Mutant #2, and mutant #41 were appended either to the N terminus or C terminus of AcGFP and analyzed using the cell-based screening assay described previously to identify destabilizing domains. Cells were split into two treatment groups and incubated with either Sildenafil (10 μM) or vehicle alone for 24 hours. Cells that were mock transduced or transduced with the N terminal fused wild-type PDE5A or PDE5AN (#40) constructs were also included in the analysis. The results are presented in Table 8 where the C terminal PDE5A mutants are denoted as PDE5AC and N terminal PDE5A mutants are denoted as PDE5AN.

TABLE 8
N-terminal Vs. C-terminal PDE5A derived DD

| | Mean FITC-A | |
|---|---|---|
| | | Sildenafil |
| Construct | DMSO | (10 μM) |
| 3T3 Parental cells | 571 | — |
| PDE5AN (#2) | 1271 | 2298 |
| PDE5AC (#2) | 1342 | 5030 |
| PDE5AN (#41) | 1284 | 1473 |
| PDE5AC (#41) | 1443 | 4620 |
| PDE5AN (WT) | 7519 | 8012 |
| PDE5AN (#40) | 1438 | 1949 |

The stabilization ratio was calculated as the fold change in GFP intensity of the AcGFP-PDE5A construct in Sildenafil treated sample compared to vehicle alone treated sample. The ratios are presented in Table 9.

TABLE 9
PDE5 DD comparative analysis

| | Stabilization Ratio | |
|---|---|---|
| | N terminus | C terminus |
| Construct | fusion | fusion |
| #2 | 1.8 | 3.75 |
| #41 | 1.15 | 3.2 |

As shown in Table 9, the stabilization ratios obtained with C terminus fusion proteins was much higher that N terminus fusion proteins. Construct #2 however did show modest ligand dependent stabilization when appended to the N terminus of the protein of interest. These data show that the ability of a particular DD mutant to stabilize or destabilize a protein of interest may depend on the positioning of the DD with respect to the protein.

### Example 11. Response of PDE5A-Derived Destabilizing Domains to Vardenafil, Tadalafil and Sildenafil

Mutations were created in PDE5A (535-860) catalytic domain using site directed mutagenesis and the mutants were cloned as AcGFP fusion into pELNS vector carrying mCherry marker and packaged in lentiviral vectors. HCT116 cells were transduced with the constructs and stable integrants were selected by cell sorting using mCherry marker. Cells were treated with vehicle (dimethyl sulfoxide, DMSO) or 10 μM Vardenafil (VDF) for 48 hours. Following the treatment, cells were analyzed for GFP expression using FACS. Basal expression was calculated as the ratio of GFP signal in the absence of ligand for a given cell line to auto fluorescence of parental HCT-116 cells. Stabilization ratio was calculated as the ratio of the GFP signal observed in the presence of stabilizing ligand to the signal from the same cell line in the absence of ligand. The results are provided in Table 10.

TABLE 10
Response to Vardenafil

| | Basal | Stabilization |
|---|---|---|
| Construct | (DMSO/Parental) | ratio |
| PDE5AN (#53) | 14.0 | 6.1 |
| PDE5AC (#13) | 11.3 | 7.0 |
| PDE5AC (#25) | 7.1 | 10.1 |
| PDE5AC (#28) | 10.4 | 6.5 |
| PDE5AC (#42) | 20.0 | 6.0 |
| PDE5AC (#43) | 8.7 | 7.4 |
| PDE5AC (#69) | 6.5 | 6.8 |

As shown in Table 10, PDE5AC (#25), PDE5AC (#28), PDE5AC (#42), and PDE5AC (#43) showed stabilization ratios with Vardenafil that were greater than the stabilization ratios obtained with sildenafil in similar experiments (see Table 2 and Table 6), indicating that Vardenafil stabilization of PDE5A DDs is more potent that sildenafil. For PDE5AN (#3) PDE5AN (#53), PDE5AC (#13) and PDE5AC (#69), stabilization ratios similar to those observed for sildenafil were obtained (see Table 2 and Table 6) indicating that these PDE5A DDs may be stabilized by multiple ligands. All mutants tested also showed some basal expression in the absence of ligand. PDE5AC (#69) and PDE5AC (#25) showed the lowest basal expression levels among the mutants tested.

HCT 116 cells transduced with the PDE5A DD derived GFP constructs were also tested with increasing doses of Vardenafil. The HCT116 cells were incubated for 48 hours with Vardenafil starting at a dose of 0.002 μM to 30 μM. The GFP intensity of the Vardenafil treated samples was compared to the parental untransduced HCT116 and DMSO treated cells for each construct. The mean fluorescence intensity was calculated for each sample and used to calculate the stabilization ratios as well as the basal expression for each construct. The GFP expression was measured using flow cytometry and the mean fluorescence intensity (MFI) was calculated for each sample. The MFI values are shown in Table 11 and Table 12. The stabilization ratios are shown in Table 13 and Table 14 and the basal expression is show in Table 15.

TABLE 11
Response to Vardenafil

| Vardenafil | PDE5AN | PDE5AN | PDE5AC | PDE5AC |
|---|---|---|---|---|
| (μM) | (#3) | (#53) | (#13) | (#25) |
| DMSO | 8934 | 14196 | 14659 | 9555 |
| 0.002 | 8767 | 14117 | 18393 | 9737 |
| 0.005 | 9620 | 14485 | 27661 | 9694 |
| 0.014 | 10487 | 15735 | 59673 | 9767 |
| 0.041 | 14086 | 17260 | 78117 | 10468 |
| 0.123 | 20568 | 25577 | 88571 | 13348 |
| 0.370 | 36786 | 43744 | 99160 | 20522 |
| 1.111 | 66543 | 81455 | 99377 | 38958 |
| 3.333 | 98986 | 131016 | 105500 | 66337 |
| 10 | 119591 | 173576 | 97493 | 94642 |
| 30 | 131667 | 188519 | 105335 | 120927 |

TABLE 12
Response to Vardenafil

| Vardenafil | PDE5AC | PDE5AC | PDE5AC | PDE5AC |
|---|---|---|---|---|
| (μM) | (#28) | (#42) | (#43) | (#69) |
| DMSO | 13817 | 25111 | 12047 | 9466 |
| 0.002 | 15437 | 25236 | 13329 | 10817 |
| 0.005 | 19446 | 28309 | 17702 | 13682 |
| 0.014 | 29314 | 33958 | 33483 | 27457 |
| 0.041 | 45804 | 46882 | 55300 | 32264 |
| 0.123 | 74153 | 81233 | 70385 | 49884 |
| 0.370 | 81663 | 114847 | 78215 | 74730 |
| 1.111 | 89426 | 136303 | 83052 | 62065 |
| 3.333 | 92290 | 147512 | 88707 | 66625 |
| 10 | 86985 | 161587 | 85867 | 64380 |
| 30 | 93145 | 199877 | 86318 | 22775 |

TABLE 13
Stabilization ratio with Vardenafil

| Vardenafil | PDE5AN | PDE5AN | PDE5AC | PDE5AC |
|---|---|---|---|---|
| (μM) | (#3) | (#53) | (#13) | (#25) |
| DMSO | — | — | — | — |
| 0.002 | 1.0 | 1.0 | 1.3 | 1.0 |
| 0.005 | 1.1 | 1.0 | 1.9 | 1.0 |
| 0.014 | 1.2 | 1.1 | 4.1 | 1.0 |
| 0.041 | 1.6 | 1.2 | 5.3 | 1.1 |
| 0.123 | 2.3 | 1.8 | 6.0 | 1.4 |
| 0.370 | 4.1 | 3.1 | 6.8 | 2.1 |
| 1.111 | 7.4 | 5.7 | 6.8 | 4.1 |
| 3.333 | 11.1 | 9.2 | 7.2 | 6.9 |
| 10 | 13.4 | 12.2 | 6.7 | 9.9 |
| 30 | 14.7 | 13.3 | 7.2 | 12.7 |

TABLE 14
Stabilization ratio with Vardenafil

| Vardenafil | PDE5AC | PDE5AC | PDE5AC | PDE5AC |
|---|---|---|---|---|
| (μM) | (#28) | (#42) | (#43) | (#69) |
| DMSO | — | — | — | — |
| 0.002 | 1.1 | 1.0 | 1.1 | 1.1 |
| 0.005 | 1.4 | 1.1 | 1.5 | 1.4 |
| 0.014 | 2.1 | 1.4 | 2.8 | 2.9 |
| 0.041 | 3.3 | 1.9 | 4.6 | 3.4 |
| 0.123 | 5.4 | 3.2 | 5.8 | 5.3 |
| 0.370 | 5.9 | 4.6 | 6.5 | 7.9 |
| 1.111 | 6.5 | 5.4 | 6.9 | 6.6 |
| 3.333 | 6.7 | 5.9 | 7.4 | 7.0 |
| 10 | 6.3 | 6.4 | 7.1 | 6.8 |
| 30 | 6.7 | 8.0 | 7.2 | 2.4 |

TABLE 15
Basal expression with Vardenafil

| | MFI | |
|---|---|---|
| Construct | (with DMSO) | Basal |
| Parental | 1954 | — |
| PDE5AN (#3) | 8934 | 4.57 |
| PDE5AN (#53) | 14196 | 7.27 |
| PDE5AC (#13) | 14659 | 7.50 |
| PDE5AC (#25) | 9555 | 4.89 |
| PDE5AC (#28) | 13817 | 7.07 |
| PDE5AC (#42) | 25111 | 12.85 |
| PDE5AC (#43) | 12047 | 6.17 |
| PDE5AC (#69) | 9466 | 4.84 |

All mutants tested showed a dose dependent increase in the expression of GFP. As shown in Table 13 and Table 14, PDE5AC (#13), PDE5AN (#3), PDE5AC (#28), PDE5AC (#42) PDE5AC (#43), and PDE5AC (#69) showed ligand dependent stabilization with as little as 0.005 μM Vardenafil, as indicated by the greater than 1 stabilization ratios obtained with those constructs. PDE5AN (#3) showed the highest stabilization ratio at maximum ligand dose of 30 μM indicating that this construct showed maximum ligand dependent stabilization among the constructs tested. As shown in Table 15, the lowest basal expression was obtained with PDE5AN (#3) which was also the construct that showed the highest stabilization ratio at highest concentration of ligand tested. These data indicate that PDE5AN (#3) has the largest dynamic range among the constructs tested with Vardenafil.

PDE5AN (#3) was cloned as AcGFP fusion into pELNS vector carrying mCherry marker and packaged in lentiviral vectors. HCT116 cells were transduced with the construct and stable integrants were selected by cell sorting using mCherry marker. Cells were treated with vehicle (dimethyl sulfoxide, DMSO) or varying doses of Tadalafil (TDF) for 48 hrs with doses ranging from 0.01 μM to 100 μM. Following the treatment, cells were analyzed for GFP expression using FACS. Basal expression was calculated as the ratio of GFP signal in the absence of ligand for a given cell line to auto fluorescence of parental HCT-116 cells. Stabilization ratio was calculated as the ratio of the GFP signal observed in the presence of stabilizing ligand to the signal from the same cell line in the absence of ligand. The results are provided in Table 16.

TABLE 16
Response to Tadalafil

| Tadalafil | | Stabilization |
|---|---|---|
| (μM) | MFI | ratio |
| Parental | 1175 | — |
| DMSO | 6944 | — |
| 0.01 | 6781 | 1.0 |
| 0.02 | 6685 | 1.0 |
| 0.05 | 7013 | 1.0 |
| 0.14 | 6988 | 1.0 |
| 0.41 | 7648 | 1.1 |
| 1.23 | 8973 | 1.3 |
| 3.7 | 14097 | 2.0 |
| 11.11 | 26560 | 3.8 |
| 33.33 | 49889 | 7.2 |
| 100 | 68532 | 9.9 |

As shown in Table 16, PDE5AN (#3), a Tadalafil dose dependent increase in the MFI was observed. Stabilization ratios greater than 1 were obtained with Tadalafil at concentrations greater than 0.41 (μM), indicating ligand dependent stabilization of PDE5AN (#3) with sub micromolar concentrations of Tadalafil.

HCT 116 cells transduced with the PDE5A DD derived GFP constructs were also tested with increasing doses of Tadalafil. The HCT116 cells were incubated for 48 hours with Tadalafil starting at dose 0.01 μM to 100 μM. The GFP intensity of the Tadalafil treated samples was compared to the parental untransduced HCT116 and DMSO treated cells for each construct. The mean fluorescence intensity was calculated for each sample and used to calculate the stabilization ratios as well as the basal expression for each construct. The MFI values are shown in Table 17 and Table 18. The stabilization ratios are shown in Table 19 and Table 20 and the basal expression is show in Table 21.

TABLE 17
Response to Tadalafil

| Tadalafil | PDE5AN | PDE5AC | PDE5AC |
|---|---|---|---|
| (μM) | (#53) | (#13) | (#25) |
| DMSO | 12071 | 14416 | 9297 |
| 0.01 | 11635 | 14388 | 9233 |
| 0.02 | 11690 | 14908 | 8860 |
| 0.05 | 12100 | 16488 | 9404 |
| 0.14 | 12331 | 21292 | 9478 |
| 0.41 | 13422 | 33396 | 9858 |
| 1.23 | 16255 | 58867 | 12432 |
| 3.7 | 24201 | 81755 | 19294 |
| 11.11 | 42644 | 92484 | 31763 |
| 33.33 | 80088 | 96959 | 54151 |
| 100 | 104032 | 105048 | 70389 |

TABLE 18
Response to Tadalafil

| Tadalafil | PDE5AC | PDE5AC | PDE5AC | PDE5AC |
|---|---|---|---|---|
| (μM) | (#28) | (#42) | (#43) | (#69) |
| DMSO | 15004 | 22601 | 10998 | 8822 |
| 0.01 | 14073 | 22787 | 11444 | 9337 |
| 0.02 | 14513 | 23274 | 11479 | 10353 |
| 0.05 | 15730 | 23278 | 11764 | 12365 |
| 0.14 | 18316 | 24532 | 13429 | 19039 |
| 0.41 | 28462 | 26765 | 18940 | 32821 |
| 1.23 | 44739 | 35325 | 32204 | 52033 |
| 3.7 | 69430 | 59821 | 49646 | 62558 |
| 11.11 | 85998 | 92400 | 64186 | 69440 |
| 33.33 | 95189 | 122658 | 74446 | 68289 |
| 100 | 97016 | 139002 | 76281 | 67259 |

TABLE 19
Stabilization ratio with Tadalafil

| Tadalafil | PDE5AN | PDE5AC | PDE5AC |
|---|---|---|---|
| (μM) | (#53) | (#13) | (#25) |
| DMSO | — | — | — |
| 0.01 | 1.0 | 1.0 | 1.0 |
| 0.02 | 1.0 | 1.0 | 1.0 |
| 0.05 | 1.0 | 1.1 | 1.0 |
| 0.14 | 1.0 | 1.5 | 1.0 |
| 0.41 | 1.1 | 2.3 | 1.1 |
| 1.23 | 1.3 | 4.1 | 1.3 |
| 3.7 | 2.0 | 5.7 | 2.1 |
| 11.11 | 3.5 | 6.4 | 3.4 |
| 33.33 | 6.6 | 6.7 | 5.8 |
| 100 | 8.6 | 7.3 | 7.6 |

TABLE 20
Stabilization ratio with Tadalafil

| Tadalafil | PDE5AC | PDE5AC | PDE5AC | PDE5AC |
|---|---|---|---|---|
| (μM) | (#28) | (#42) | (#43) | (#69) |
| DMSO | — | — | — | — |
| 0.01 | 0.9 | 1.0 | 1.0 | 1.1 |
| 0.02 | 1.0 | 1.0 | 1.0 | 1.2 |
| 0.05 | 1.0 | 1.0 | 1.1 | 1.4 |
| 0.14 | 1.2 | 1.1 | 1.2 | 2.2 |
| 0.41 | 1.9 | 1.2 | 1.7 | 3.7 |
| 1.23 | 3.0 | 1.6 | 2.9 | 5.9 |
| 3.7 | 4.6 | 2.6 | 4.5 | 7.1 |
| 11.11 | 5.7 | 4.1 | 5.8 | 7.9 |
| 33.33 | 6.3 | 5.4 | 6.8 | 7.7 |
| 100 | 6.5 | 6.2 | 6.9 | 7.6 |

TABLE 21
Basal expression with Tadalafil

| Tadalafil | MFI | Basal |
|---|---|---|
| (μM) | (with DMSO) | Expression |
| Parental | 1280 | — |
| PDE5AN (#53) | 12071 | 9.43 |
| PDE5AC (#13) | 14416 | 11.26 |
| PDE5AC (#25) | 9297 | 7.26 |
| PDE5AC (#28) | 15004 | 11.72 |
| PDE5AC (#42) | 22601 | 17.66 |
| PDE5AC (#43) | 10998 | 8.59 |
| PDE5AC (#69) | 8822 | 6.89 |

All mutants tested showed a dose dependent increase in the expression of GFP. As shown in Table 19 and Table 20, PDE5AC (#13), and PDE5AC (#69) showed ligand dependent stabilization with as little as 0.14 μM Tadalafil, as indicated by the greater than 1.5 stabilization ratios obtained with those constructs. PDE5AN (#25) and PDE5AC (#69) showed the highest stabilization ratio at maximum ligand dose of 100 μM indicating that this construct showed maximum ligand dependent stabilization among the constructs tested. As shown in Table 21, the lowest basal expression was obtained with PDE5AC (#69) which was also one of the constructs that showed the highest stabilization ratio at highest concentration of ligand tested. These data indicate that PDE5AC (#69) has the largest dynamic range among the constructs tested with Tadalafil.

Similar experiments were performed in HCT116 cells using sildenafil. The above-mentioned DDs were cloned as AcGFP fusion into pELNS vector carrying mCherry marker and packaged in lentiviral vectors. HCT116 cells were transduced with the constructs and stable integrants were selected by cell sorting using mCherry marker. Cells were treated with sildenafil at the following μM concentrations: 0.01, 0.02, 0.05, 0.14, 0.41, 1.23, 3.7, 11.11, 33.33, and 100 for a duration of 48 hours. The fluorescence was analyzed by flow cytometry and the mean fluorescence intensity was calculated for all samples. All clones tested showed a dose dependent increase in expression. PDE5AC (#28) showed the highest MFI values upon treatment with 100 μM sildenafil. The MFI values are shown in Table 22 and Table 23. The stabilization ratios are shown in Table 24 and Table 25 and the basal expression is show in Table 26.

TABLE 22
Response to Sildenafil

| Sildenafil | PDE5AN | PDE5AN | PDE5AC | PDE5AC |
|---|---|---|---|---|
| (μM) | (#3) | (#53) | (#13) | (#25) |
| DMSO | 9835 | 13747 | 15122 | 10217 |
| 0.01 | 9888 | 12801 | 14787 | 10169 |
| 0.02 | 10157 | 14045 | 17318 | 9354 |
| 0.05 | 9952 | 13801 | 26905 | 10120 |
| 0.14 | 10475 | 14048 | 48611 | 10128 |
| 0.41 | 12302 | 16175 | 69042 | 10207 |
| 1.23 | 16826 | 20410 | 84772 | 12328 |
| 3.70 | 27542 | 30372 | 82519 | 17466 |
| 11.11 | 52641 | 59723 | 92732 | 26947 |
| 33.33 | 85509 | 99558 | 93336 | 46897 |
| 100.00 | 103938 | 117792 | 76090 | 51774 |

TABLE 23
Response to Sildenafil

| Sildenafil | PDE5AC | PDE5AC | PDE5AC | PDE5AC |
|---|---|---|---|---|
| (μM) | (#28) | (#42) | (#43) | (#69) |
| DMSO | 13208 | 23943 | 11147 | 9575 |
| 0.01 | 14613 | 23295 | 11962 | 9574 |
| 0.02 | 16502 | 23606 | 11940 | 11755 |
| 0.05 | 17801 | 24817 | 15203 | 11944 |
| 0.14 | 26694 | 29495 | 24319 | 19733 |
| 0.41 | 43862 | 40817 | 40470 | 30090 |
| 1.23 | 65762 | 63410 | 57723 | 44180 |
| 3.70 | 83657 | 91798 | 73400 | 52782 |
| 11.11 | 80269 | 127187 | 76900 | 59723 |
| 33.33 | 83663 | 149906 | 94863 | 65183 |
| 100.00 | 80213 | 164269 | 56552 | 51774 |

TABLE 24
Stabilization ratio with Sildenafil

| Sildenafil | PDE5AN | PDE5AN | PDE5AC | PDE5AC |
|---|---|---|---|---|
| (μM) | (#3) | (#53) | (#13) | (#25) |
| DMSO | — | — | — | — |
| 0.01 | 1.0 | 0.9 | 1.0 | 1.0 |
| 0.02 | 1.0 | 1.0 | 1.1 | 0.9 |
| 0.05 | 1.0 | 1.0 | 1.8 | 1.0 |
| 0.14 | 1.1 | 1.0 | 3.2 | 1.0 |
| 0.41 | 1.3 | 1.2 | 4.6 | 1.0 |
| 1.23 | 1.7 | 1.5 | 5.6 | 1.2 |
| 3.70 | 2.8 | 2.2 | 5.5 | 1.7 |
| 11.11 | 5.4 | 4.3 | 6.1 | 2.6 |
| 33.33 | 8.7 | 7.2 | 6.2 | 4.6 |
| 100.00 | 10.6 | 8.6 | 5.0 | 5.1 |

TABLE 25
Stabilization ratio with Sildenafil

| Sildenafil | PDE5AC | PDE5AC | PDE5AC | PDE5AC |
|---|---|---|---|---|
| (μM) | (#28) | (#42) | (#43) | (#69) |
| DMSO | — | — | — | — |
| 0.01 | 1.1 | 1.0 | 1.1 | 1.0 |
| 0.02 | 1.2 | 1.0 | 1.1 | 1.2 |
| 0.05 | 1.3 | 1.0 | 1.4 | 1.2 |
| 0.14 | 2.0 | 1.2 | 2.2 | 2.1 |
| 0.41 | 3.3 | 1.7 | 3.6 | 3.1 |
| 1.23 | 5.0 | 2.6 | 5.2 | 4.6 |
| 3.70 | 6.3 | 3.8 | 6.6 | 5.5 |
| 11.11 | 6.1 | 5.3 | 6.9 | 6.2 |
| 33.33 | 6.3 | 6.3 | 8.5 | 6.8 |
| 100.00 | 6.1 | 6.9 | 5.1 | 5.4 |

TABLE 26
Basal expression with Sildenafil

| | MFI | Basal |
|---|---|---|
| Description | (with DMSO) | expression |
| Parental | 1287 | — |
| PDE5AN (#3) | 9835 | 7.6 |
| PDE5AN (#53) | 13747 | 10.7 |
| PDE5AC (#13) | 15122 | 11.7 |
| PDE5AC (#25) | 10217 | 7.9 |
| PDE5AC (#28) | 13208 | 10.3 |
| PDE5AC (#42) | 23943 | 18.6 |
| PDE5AC (#43) | 11147 | 8.7 |
| PDE5AC (#69) | 9575 | 7.4 |

All mutants tested showed a dose dependent increase in the expression of GFP. As shown in Table 22 and Table 23, PDE5AC (#13), showed ligand dependent stabilization with as little as 0.05 μM Sildenafil, as indicated by the greater than 1.5 stabilization ratios obtained with that construct. PDE5AN (#28), PDE5AC (#43) and PDE5AC (#69) showed stabilization ratios greater than 1.5 with 0.14 μM of Sildenafil. PDE5AN (#3) showed the highest stabilization ratio at maximum ligand dose of 100 μM indicating that this construct showed maximum ligand dependent stabilization among the constructs tested. As shown in Table 26, the lowest basal expression was obtained with PDE5AC (#69). These data indicate that the PDE5A DDs described herein can be stabilized by multiple ligands.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A destabilizing domain, comprising a region or a portion of human PDE5A wherein the stability-affecting protein domain is identified by introducing a predetermined amino acid into one or more selected sequence positions in a predefined region of the protein by walk through mutagenesis.

2. The destabilizing domain of claim 1, wherein the predefined region comprises the catalytic domain of PDE5A.

3. The destabilizing domain of claim 2, wherein the predefined region is selected from a position 535-541, a position 555-564; a position 587-591; a position 604-609; a position 630-636; a position 674-678; a position 762-766; a position 795-797; and a position 809-816 of human PDE5A (SEQ ID NO. 1).

4. The destabilizing domain of claim 3, wherein the predetermined amino acid is introduced into one or more predefined regions.

5. The destabilizing domain of claim 4, wherein the predetermined amino acid is absent or selected from an alanine, an arginine, an asparagine, an aspartic acid, a cysteine, a glutamine, a glutamic acid, a glycine, a histidine, an isoleucine, a leucine, a lysine, a methionine, a phenylalanine, a proline, a serine, a threonine, a tryptophan, a tyrosine, and a valine.

6. The destabilizing domain of claim 5, wherein multiple predetermined amino acids are introduced into one or more predefined regions, and wherein the destabilization domain comprises an amino acid sequence selected from the group consisting of SEQ ID NO: 24, SEQ ID NO: 25, SEQ ID NOs: 28, SEQ ID NO: 29, SEQ ID NO: 30, SEQ ID NO: 35, SEQ ID NO: 66, and SEQ ID NO: 67.


SEQUENCE LISTING (77 sequences)

SEQ ID NO: 1 - protein, 875 aa, Homo sapiens
MERAGPSFGQQRQQQQPQQQKQQQRDQDSVEAWLDDHWDFTFSYFVRKATREMVNAWFAERVHTIPVCKEGIRGHTESCSCPLQQSPRADNSAPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 2 - DNA, 2628 bp, Homo sapiens
  source 1..2628: mol_type = other DNA
atggagcgggccggccccagcttcgggcagcagcgacagcagcagcagccccagcagcagaagcagcagcagagggatcaggactcggtcgaagcatggctggacgatcactgggactttaccttctcatactttgttagaaaagccaccagagaaatggtcaatgcatggtttgctgagagagttcacaccatccctgtgtgcaaggaaggtatcagaggccacaccgaatcttgctcttgtcccttgcagcagagtcctcgtgcagataacagtgcccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactga

SEQ ID NO: 3 - protein, 833 aa, Homo sapiens
MLPFGDKTREMVNAWFAERVHTIPVCKEGIRGHTESCSCPLQQSPRADNSAPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 4 - DNA, 6836 bp, Homo sapiens
  source 1..6836: mol_type = other DNA
tggttctgagtcgccgacctcacctcacctcacctgcgcgcgttgttgcccctacggagcctcctggagtccaggatcggcggagttcgaaaacgaacttcccacgtttgctatgttgccctttggagacaaaacaagagaaatggtcaatgcatggtttgctgagagagttcacaccatccctgtgtgcaaggaaggtatcagaggccacaccgaatcttgctcttgtcccttgcagcagagtcctcgtgcagataacagtgcccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactgagtggcctatttcatgcagagttgaagtttacagagatggtgtgttctgcaatatgcctagtttcttacacactgtctgtatagtgtctgtatttggtatatactttgccactgctgtatttttatttttgcacaacttttgagagtatagcatgaatgtttttagaggactattacatattttttgtatatttgttttatgctactgaactgaaaggatcaacaacatccactgttagcacattgataaaagcattgtttgtgatatttcgtgtactgcaaagtgtatgcagtattcttgcactgaggtttttttgcttggggattattttaaataattggtttttgtgttttctgaattaccattttttcaagaatgtttggaatctttcctttttcaaaagtaggttaggagcaaattatcatacattctgtgacatttaaagcctttataggatagtgaaaaatgctggctgagtggattttaagagaaataattgtatttgttaacagtgtctttttttaaaaagttaaggcactctgaaacaaatggaaagtcctatgaaactgtattgtaaagaaaacattatttaattgatatgctgttttgtgagagaacaggcaagacagaactttgtcacttcagtgcagtacatttttctgaaagctacccataaaatcactttcatctcacctacctgatgcaaagcaggtgaaaccttaggagatgatccagtcactgacttgattgagggataagtgtgatttagaaatggaatggccttggatgtctatcagtgaagaaaaatgttctgttagaagatctctctaagagtttttttccttctgagcttccttttcaaaataaaagtgacaattgtagcattgacttgaagtgagacatggttatagataagagagtacaaaatgactctttttcctgtcaattgaaatttaaagaaaagttttaattatataaatagcaaagggctattgccaatactagggtcaaaaatgaatttgagggaacagtgggtaagaaactttatgcctgaataacatttagcagtattgtgattgaaaaattgccatattttgatgtataggacaagtcaactgagatccagagaatcctggatgtgaatgctaaacactggcccttaactcacattcaatgtattttcttcccataacatttagtatagttaatattttcttagaatttgagcccatttaagtggattaatattctacatgtgtgcccctaaagacacatttactcaatattggagaagtagataatgaattaagcaactggtctaggaaaggaaaatttgtttcaaatatgcaggaatgtttggatttggggagagtagaaggagagatttgcttgatttgttaacttctacctccaacccacaaaaaagatatttgatctgagtttctatcactaatttggatagaaaatttctaagggacatggtaatccagcattctcaaggacctttcgccaaaatgtgttttccatctatgtcccgattcccctaaattttgcctaaaattcagtatgttccttaagtttttaaaattctgagtgtgtacaaatatcttgacataatgcagttttatttttatcattctggtaaaaaaacaaaaaatagaagcaaaacacattgtattgccattattttgtatttggtaaaggttaatctaggaagttaccaactgtttaatgctatatgtattgtatacttgtattttcaggatattttattttttttgccatacagataaaatttgtaaggttgcccctttgtggcactggtgtgtaaaatacacagactatcactaaaataatagttatatatacatacaggtgtatacttatgcatgcatacataaatccttagtatagaaaaattgcataaagaatagcaatctttaataaacctttttattacattgtgatttagcagttatgctaaaatatgtacttatgctttagtagtttgtttggtcccctctagtatgtgtcactgagaaattttttaaagacatggtagatcgtgtttagaggctttgtatgtgtgtcattttaataagcaagaagatatatttagattagaaatggtttggtctgcctttgaatattgtttattttactttactagttgagacattaaaggaagctgggcaatgcctattttatttctttgttggatattttagttcatacaaagcagagtacttctttagggctggttaattggttcaaataatttttaatttcctttctagtatcttctcaagttggaaaaatatacatacagtcctccttcaccttactctgtatttatattacccataactagcaagaagttcttgttctagattttttgtttgtttagttataacagagtaacataccatttaattacaatttttagccagaaaagtccccactattttactaacttgttaaaagatatctatataattgcctggccttatatttttcagtagattagaccgtgccaatcacaatcctgggtggatttgtgtaagtcacttaacctctgtgtgcctaaacaagttgtgcttttttaaaaggagttatgtttgggcaaagcctttgtcttcaagcagaatgtcacagaaggcagctactttataagccccaatgggccatggagaccactgtcagaaatgggatattagtctagagagaaggtgatctattcccacatgtcatttctaatgttgagtttccatgactgaacaaagagaatatatttattcagcttcacttgcagatcactagtgaatgtgagatttagagctcattgagtatattgcttcaaggtacaaacccaggatgatgatgttgtcaccactgtctcttaattttgaataatagtttcctttaataggagtattagagataagaaagtatatgaaaatatactggaaatattggattcttggagaaaactgttcagtcacagatatattcttgcctagcagtgaagtgcctttattttcagcatagcaaataaatattagacctgttccaatttgatctacaatttttttctgtgtttttcaccagattgtactcctaaaacttaacaggccatcacaagcaattgtcttttgtttacaagattgatttaatatgagaggatacaaaatgtcatcgttatcctctcttatgaacaactgtagtcaaaataaggtggcacaatttaattgttttgtatcagaaatacactgacccaccttttattgagtcctgccacatgttaggtaccgtgctctgctgtggagacagagcagtgaccccaaggagctcacggtccctgaaggaggtgctagagaagagacttagcttctgatactgccaatttaatgtgagaacatggggtatactgcatcatttccattttcatcaataacatatgttttatgcaccttctttacctgaaacttactaagaatctaccagtaaacaaacatcctgtctttttgcaagtatgaatcacttaacctgctgatagttgaagaacactttaggagttttgtattcttgtatatagtttattttttccatgtgctagccaggtaaagattacacagttcttctggactgttaaattgtgcatggttttggaccccttctgctctactacagagagtgaagaagaaagtattaaagctcactttaccattccatatacttactaaaagcctgtgtaaacatgtcttaatgaatgttgttgaaagcaatgtaaatagttgaaaatataaatttatattacagtttaagaaaaccttatgaggcatcactagccactgttaatatctatttgtattcttataccttttcaatatatttgaacaaatatagtttctggcactatttttatactaggaaaaggagttactatgtatattatgcttagcttttaaggcattttaaataaccatgaatgttgatttcattactttcctttcctccatcacgagagtcatttcagatgactctttcatgacaaaatcactttaaaggaacacttacctccgattcctgtataaagtcatgagatggtcaaggtggttttccattgtgcaaattcttcacctgtcagtggtttcctcattttgccatgctttgtaaaaataaaaagaatgatcaagtaggtatgaaaaaaaaaaaaaaaaaa

SEQ ID NO: 5 - protein, 823 aa, Homo sapiens
MVNAWFAERVHTIPVCKEGIRGHTESCSCPLQQSPRADNSAPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 6 - DNA, 6788 bp, Homo sapiens
  source 1..6788: mol_type = other DNA
cctcgccctggccctggctgggcggggaagctggggtgagggacgagtccacgggacagcccaaaggcaacatgacggaaccttgccaaaagaaatggtcaatgcatggtttgctgagagagttcacaccatccctgtgtgcaaggaaggtatcagaggccacaccgaatcttgctcttgtcccttgcagcagagtcctcgtgcagataacagtgcccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactgagtggcctatttcatgcagagttgaagtttacagagatggtgtgttctgcaatatgcctagtttcttacacactgtctgtatagtgtctgtatttggtatatactttgccactgctgtatttttatttttgcacaacttttgagagtatagcatgaatgtttttagaggactattacatattttttgtatatttgttttatgctactgaactgaaaggatcaacaacatccactgttagcacattgataaaagcattgtttgtgatatttcgtgtactgcaaagtgtatgcagtattcttgcactgaggtttttttgcttggggattattttaaataattggtttttgtgttttctgaattaccattttttcaagaatgtttggaatctttcctttttcaaaagtaggttaggagcaaattatcatacattctgtgacatttaaagcctttataggatagtgaaaaatgctggctgagtggattttaagagaaataattgtatttgttaacagtgtctttttttaaaaagttaaggcactctgaaacaaatggaaagtcctatgaaactgtattgtaaagaaaacattatttaattgatatgctgttttgtgagagaacaggcaagacagaactttgtcacttcagtgcagtacatttttctgaaagctacccataaaatcactttcatctcacctacctgatgcaaagcaggtgaaaccttaggagatgatccagtcactgacttgattgagggataagtgtgatttagaaatggaatggccttggatgtctatcagtgaagaaaaatgttctgttagaagatctctctaagagtttttttccttctgagcttccttttcaaaataaaagtgacaattgtagcattgacttgaagtgagacatggttatagataagagagtacaaaatgactctttttcctgtcaattgaaatttaaagaaaagttttaattatataaatagcaaagggctattgccaatactagggtcaaaaatgaatttgagggaacagtgggtaagaaactttatgcctgaataacatttagcagtattgtgattgaaaaattgccatattttgatgtataggacaagtcaactgagatccagagaatcctggatgtgaatgctaaacactggcccttaactcacattcaatgtattttcttcccataacatttagtatagttaatattttcttagaatttgagcccatttaagtggattaatattctacatgtgtgcccctaaagacacatttactcaatattggagaagtagataatgaattaagcaactggtctaggaaaggaaaatttgtttcaaatatgcaggaatgtttggatttggggagagtagaaggagagatttgcttgatttgttaacttctacctccaacccacaaaaaagatatttgatctgagtttctatcactaatttggatagaaaatttctaagggacatggtaatccagcattctcaaggacctttcgccaaaatgtgttttccatctatgtcccgattcccctaaattttgcctaaaattcagtatgttccttaagtttttaaaattctgagtgtgtacaaatatcttgacataatgcagttttatttttatcattctggtaaaaaaacaaaaaatagaagcaaaacacattgtattgccattattttgtatttggtaaaggttaatctaggaagttaccaactgtttaatgctatatgtattgtatacttgtattttcaggatattttattttttttgccatacagataaaatttgtaaggttgcccctttgtggcactggtgtgtaaaatacacagactatcactaaaataatagttatatatacatacaggtgtatacttatgcatgcatacataaatccttagtatagaaaaattgcataaagaatagcaatctttaataaacctttttattacattgtgatttagcagttatgctaaaatatgtacttatgctttagtagtttgtttggtcccctctagtatgtgtcactgagaaattttttaaagacatggtagatcgtgtttagaggctttgtatgtgtgtcattttaataagcaagaagatatatttagattagaaatggtttggtctgcctttgaatattgtttattttactttactagttgagacattaaaggaagctgggcaatgcctattttatttctttgttggatattttagttcatacaaagcagagtacttctttagggctggttaattggttcaaataatttttaatttcctttctagtatcttctcaagttggaaaaatatacatacagtcctccttcaccttactctgtatttatattacccataactagcaagaagttcttgttctagattttttgtttgtttagttataacagagtaacataccatttaattacaatttttagccagaaaagtccccactattttactaacttgttaaaagatatctatataattgcctggccttatatttttcagtagattagaccgtgccaatcacaatcctgggtggatttgtgtaagtcacttaacctctgtgtgcctaaacaagttgtgcttttttaaaaggagttatgtttgggcaaagcctttgtcttcaagcagaatgtcacagaaggcagctactttataagccccaatgggccatggagaccactgtcagaaatgggatattagtctagagagaaggtgatctattcccacatgtcatttctaatgttgagtttccatgactgaacaaagagaatatatttattcagcttcacttgcagatcactagtgaatgtgagatttagagctcattgagtatattgcttcaaggtacaaacccaggatgatgatgttgtcaccactgtctcttaattttgaataatagtttcctttaataggagtattagagataagaaagtatatgaaaatatactggaaatattggattcttggagaaaactgttcagtcacagatatattcttgcctagcagtgaagtgcctttattttcagcatagcaaataaatattagacctgttccaatttgatctacaatttttttctgtgtttttcaccagattgtactcctaaaacttaacaggccatcacaagcaattgtcttttgtttacaagattgatttaatatgagaggatacaaaatgtcatcgttatcctctcttatgaacaactgtagtcaaaataaggtggcacaatttaattgttttgtatcagaaatacactgacccaccttttattgagtcctgccacatgttaggtaccgtgctctgctgtggagacagagcagtgaccccaaggagctcacggtccctgaaggaggtgctagagaagagacttagcttctgatactgccaatttaatgtgagaacatggggtatactgcatcatttccattttcatcaataacatatgttttatgcaccttctttacctgaaacttactaagaatctaccagtaaacaaacatcctgtctttttgcaagtatgaatcacttaacctgctgatagttgaagaacactttaggagttttgtattcttgtatatagtttattttttccatgtgctagccaggtaaagattacacagttcttctggactgttaaattgtgcatggttttggaccccttctgctctactacagagagtgaagaagaaagtattaaagctcactttaccattccatatacttactaaaagcctgtgtaaacatgtcttaatgaatgttgttgaaagcaatgtaaatagttgaaaatataaatttatattacagtttaagaaaaccttatgaggcatcactagccactgttaatatctatttgtattcttataccttttcaatatatttgaacaaatatagtttctggcactatttttatactaggaaaaggagttactatgtatattatgcttagcttttaaggcattttaaataaccatgaatgttgatttcattactttcctttcctccatcacgagagtcatttcagatgactctttcatgacaaaatcactttaaaggaacacttacctccgattcctgtataaagtcatgagatggtcaaggtggttttccattgtgcaaattcttcacctgtcagtggtttcctcattttgccatgctttgtaaaaataaaaagaatgatcaagtaggtatgaaaaaaaaaaaaaaaaaa

SEQ ID NO: 7 - protein, 326 aa, Homo sapiens
EETRELQSLAAAVVPSAQTLKITDFSFSDFELSDLETALCTIRMFTDLNLVQNFQMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 8 - DNA, 978 bp, Homo sapiens
  source 1..978: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 9 - protein, 875 aa, synthetic construct
  REGION 1..875: note = source = /note="Description of Artificial Sequence: Synthetic construct"
MERAGPSFGQQRQQQQPQQQKQQQRDQDSVEAWLDGHWDFTFSYFVRKATREMVNAWFAERVHTIPVCKEGIRGHTESCSCPLQQSPRADNSVPGTPTRKISASEFDRPLRPIVVKDSEGTVSFLSDSEKKEQMPLTPPRFDHDEGDQCSRLLELVKDISSHLDVTALCHKIFLHIHGLISADRYSLFLVCEDSSNDKFLISRLFDVAEGSTLEEVSNNCIRLEWNKGIVGHVAALGEPLNIKDAYEDPRFNAEVDQITGYKTQSILCMPIKNHREEVVGVAQAINKKSGNGGTFTEKDEKDFAAYLAFCGIVLHNAQLYETSLLENKRNQVLLDLASLIFEEQQSLEVILKKIAATIISFMQVQKCTIFIVDEDCSDSFSSVFHMECEELEKSSDTLTREHDANKINYMYAQYVKNTMEPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 10 - DNA, 2625 bp, synthetic construct
  source 1..2625: mol_type = other DNA
atggagcgggccggccccagcttcgggcagcagcgacagcagcagcagccccagcagcagaagcagcagcagagggatcaggactcggtcgaagcatggctggacggtcactgggactttaccttctcatactttgttagaaaagccaccagagaaatggtcaatgcatggtttgctgagagagttcacaccatccctgtgtgcaaggaaggtatcagaggccacaccgaatcttgctcttgtcccttgcagcagagtcctcgtgcagataacagtgtccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgctcgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtacgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaaacctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaac

SEQ ID NO: 11 - protein, 326 aa, synthetic construct; identical to SEQ ID NO: 7
  REGION 1..326: note = source = /note="Description of Artificial Sequence: Synthetic Destabilizing Domain (DD)"

SEQ ID NO: 12 - DNA, 978 bp, synthetic construct
  misc_feature 1..978: note = Synthetic Destabilizing Domain (DD)
  source 1..978: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 13 - protein, 341 aa, synthetic construct
  REGION 1..341: note = source = /note="Description of Artificial Sequence: Synthetic Destabilizing Domain (DD)"
EETRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 14 - DNA, 1026 bp, synthetic construct
  misc_feature 1..1026: note = Synthetic Destabilizing Domain (DD)
  source 1..1026: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactga

SEQ ID NO: 15 - protein, 410 aa, synthetic construct
  REGION 1..410: note = source = /note="Description of Artificial Sequence: Synthetic Destabilizing Domain (DD)"
GVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 16 - DNA, 1233 bp, synthetic construct
  misc_feature 1..1233: note = source = /note="Description of Artificial Sequence: Synthetic Destabilizing Domain (DD)"
  source 1..1233: mol_type = other DNA
ggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactga

SEQ ID NO: 17 - protein, 456 aa, synthetic construct
  REGION 1..456: note = source = /note="Description of Artificial Sequence: Synthetic Destabilizing Domain (DD)"
EPLNIPDVSKDKRFPWTTENTGNVNQQCIRSLLCTPIKNGKKNKVIGVCQLVNKMEENTGKVKPFNRNDEQFLEAFVIFCGLGIQNTQMYEAVERAMAKQMVTLEVLSYHASAAEEETRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQEKMLINGESGQAKRN

SEQ ID NO: 18 - DNA, 1371 bp, synthetic construct
  misc_feature 1..1371: note = source = /note="Description of Artificial Sequence: Synthetic Destabilizing Domain (DD)"
  source 1..1371: mol_type = other DNA
gaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactga

SEQ ID NO: 19 - protein, 327 aa, synthetic construct
  REGION 1..327: note = source = /note="Description of Artificial Sequence: Synthetic Destabilizing Domain (DD)"
MEETRELQSLAAAVVPSAQTLKITDFSFSDFELSDLETALCTIRMFTDLNLVQNFRMKHEVLCRWILSVKKNYRKNVAYHNWRHAFNTAQCMFAALKAGEIQNKLTDLEILALLIAALSHDLDHRGVNNSYIQRSEHPLAQLYCHSIMEHHHFDQCLMILNSPGNQILSGLSIEEYKTSLKIIKQAILATDLALYIKRRGEFFELIRKNQFNLEDPHQKELFLAMLMTACDLSAITKPWPIQQRIAELVATEFFDQGDREREELNIEPTDLMNREKKNKIPSMQVGFIDAICLQLYEALTHVSEDCFPLLDGCRKNRQEWQALAEQQ

SEQ ID NO: 20 - protein, 327 aa, synthetic construct
  REGION 1..327: note = source = /note="Description of Artificial Sequence: Synthetic Destabilizing Domain (DD)"
MEGTRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDSFPLLDGCRKNRQKWQALAEQQ

SEQ ID NO: 21 - protein, 327 aa, synthetic construct
  REGION 1..327: note = source = /note="Description of Artificial Sequence: Synthetic Destabilizing Domain (DD)"
MEETRELQSLAAAVVPSAQTLKITDFSFSDFELSDLETALCTIRMFTDLNLVQSFRMKHEVLCRWILSVKKNYRENVAYHNWRHAFNTAQCMFAALKAGKIQNKLTDLEILALLIAALSHDLDHRGVSNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACVLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 22 - protein, 327 aa, synthetic construct
  REGION 1..327: note = source = /note="Description of Artificial Sequence: Synthetic Destabilizing Domain (DD)"
MEETRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQPYCHSIMEHHHFDQCLMILNSPGNQILSGLSIEEYKTTLKIIKQAILATDLALYIKRRGEFFELIRKNQFNLEDPHQKELLLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 23 - protein, 326 aa, synthetic construct
  REGION 1..326: note = source = /note="Description of Artificial Sequence: Synthetic Destabilizing Domain (DD)"
EETRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFAQCLMILNSPGNQILSGLSIEEYKTTLKIIKQAILATDLALYIKRRGEFFELIRKNQFNLEDPHQKELFLAMLMTACNLSAITKPWPIQQRIAELVATEFFDQGDRERKELNIEPTDLMNREKKNKIPCMQVGFIDAICLQLYEALTHVSEDCFPLLDGCRKNRQKWQALAEQQ

SEQ ID NO: 24 - protein, 326 aa, synthetic construct
  REGION 1..326: note = source = /note="Description of Artificial Sequence: Synthetic Destabilizing Domain (DD)"
EETRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQLDCHSIMEHHHFDQCLMILNSPGNQILSGLSIEEYKTTLKIIKQAILATDLALYIKRRGEFFEHIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 25 - protein, 326 aa, synthetic construct
  REGION 1..326: note = source = /note="Description of Artificial Sequence: Synthetic Destabilizing Domain (DD)"
EETRELQSLAAAVVPSAQTLKITDFSFSDFELSDLETALCTIRMFTDLNLVQNFRMEHEVLCRWILSVKKNYRKHVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACVLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 26 - protein, 326 aa, synthetic construct
  REGION 1..326: note = source = /note="Description of Artificial Sequence: Synthetic Destabilizing Domain (DD)"
EETRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACGLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 27 - protein, 326 aa, synthetic construct
  REGION 1..326: note = source = /note="Description of Artificial Sequence: Synthetic Destabilizing Domain (DD)"
EETRELQSLAAAVVPSAQTLKITDFSLSDFELSDLETALCTIRMFTDLNLVQNFRMKHEVLCRWILSVKENYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATNLALYIKRRGEFFELIRKNQFNLEDPHQKELFLAMLMTACDLSAITKPWPIQQRIAELVATEFFDQGDRERKEFNIEPTDLMNREKKNKIPSMQVGFIDAICLQLYEALTHVSEDCFPLLDGCRKNRQKWQALAEQQ

SEQ ID NO: 28 - protein, 326 aa, synthetic construct
  REGION 1..326: note = source = /note="Description of Artificial Sequence: Synthetic Destabilizing Domain (DD)"
DETRELQSLAAAVVPSAQTLRITDFSFSDSELSDLETALCTIRMFTDLNLVQNFRMKHEVLCRWILSVKKNYRKNVAYHNWRHAFNTAQCMFAALRAGKIQNKLTDLEILALLIAALSHDLDHRGVNNSYIQRSEHPLAQLYRHSIMEHHHFDQCLMILNSPGNQILSGLSIEEYKTTLKIIKQAILATDLALYIKRRGEFFELIRKSQFNLEDPHQKELFLAMLMTACDLSAITKPWPIQQRIAELVATEFFDQGDRERKELNTEPTDLMNREKKNKIPSAQVGFIDAICLQLYEALTHVSEDCFPLLDGCRKNRQKWQALAEQQ

SEQ ID NO: 29 - protein, 326 aa, synthetic construct
  REGION 1..326: note = source = /note="Description of Artificial Sequence: Synthetic Destabilizing Domain (DD)"
EETRELQSLAAAVVPSAQTLKITDFSFSDFELSDLETALCTIRMFTDLNLVQNFRMKHEVLCRWILSVKKNYRKHVAYHNWRHAFNTAQCMFAALKAGKIQNKLTDLEILALLIAALSHDLDHRGVNNSYIQRSEHPLAQLNCHSIMEHHHFDQCLMILNSPGNQILSGLSIEEYKTTLKIIKQAILATDLALYIKRRGEFFELIRKNQFNLEDPHQKELFLAMLMTSCDLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 30 - protein, 326 aa, synthetic construct
  REGION 1..326: note = source = /note="Description of Artificial Sequence: Synthetic Destabilizing Domain (DD)"
EETRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQSKLTDLEILALLIAALSHDLDHRGVNNSYIQRSEHPLAQLYCHSIMEHHHFAQCLMILNSPGNQILSGLSIEEYKTTLKIIKQAILATDLALYIKRRGEFFELIRKNQFNLEDPHQKELFLAMLMTACNLSAITKPWPIQQRIAELVATEFFDQGDRERKELNIEPTDLMNREKKNKIPCMQVGFIDAICLQLYEALTHVSEDCFPLLDGCRKNRQKWQALAEQQ

SEQ ID NO: 31 - protein, 326 aa, synthetic construct
  REGION 1..326: note = source = /note="Description of Artificial Sequence: Synthetic Destabilizing Domain (DD)"
EETRELQSLAAAVVPSAQTLKITDFSFSDFELSDLETALCTIRMFTDLNLVQNFRMKHEVLCRWVLSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKATLKIIKQAILATDLALYIKRRGEFFELIRKNQLNSEDPHQKELFLAMLMTACDLSAITKPWPIQQRIAELVATEFFDQGDRERKELNIEPTDPMNREKKNKIPSMQVGFIDAICLQLYEALTHVSEDCFPLLDGCRKNRQKWQALAEQQ

SEQ ID NO: 32 - protein, 326 aa, synthetic construct
  REGION 1..326: note = source = /note="Description of Artificial Sequence: Synthetic Destabilizing Domain (DD)"
EETRELQSLAAAVVPSAQTLKITDFGFSDFELSDLETALCTIRMFTDLNLAQSFRMEHEVLCRWILSVKKNYRKNVAYHNWRHAFNTAQCMFAALKAGKIQNKLTDLEILALLIAALSHDLDHRGVNNPYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDCSPLLDGCRKNRQKWQALAEQQ

SEQ ID NO: 33 - protein, 326 aa, synthetic construct
  REGION 1..326: note = source = /note="Description of Artificial Sequence: Synthetic Destabilizing Domain (DD)"
EETRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLVAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSTQVGFIDAICLQLYEALTHVSEDCFPLLDGCRKNRQKWQALAEQQ

SEQ ID NO: 34 - protein, 326 aa, synthetic construct
  REGION 1..326: note = source = /note="Description of Artificial Sequence: Synthetic Destabilizing Domain (DD)"
EETRELQSLAAAVVPSAQTLKITDFSLSDLELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATNLALYIKRRGEFFELIRKNQFNLEDPHQKELFLAMLMTACDLFAITKPWPIQQRIAELVATEFFDQGDRERKELNIEPPDLMNREKKNKIPSMQVGFIDAICLQLYEALTHVSEDCFPLLDGCRKNRQKWQALAEQQ

SEQ ID NO: 35 - protein, 326 aa, synthetic construct
  REGION 1..326: note = source = /note="Description of Artificial Sequence: Synthetic Destabilizing Domain (DD)"
EETRELRSLAAAVVPSAQTLKITDLSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQLYCRSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDATCLQLYEALTHVSEDCFPLLDGCRKNRQKWQALAEQQ

SEQ ID NO: 36 - DNA, 981 bp, synthetic construct
  misc_feature 1..981: note = source = /note="Description of Artificial Sequence: Synthetic Destabilizing Domain (DD)"
  source 1..981: mol_type = other DNA
atggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcgaaattcagaacaagctgactgacctggagatacttgcattgctgattgctgcactaagccacgatttggatcaccgtggtgtgaataactcttacatacagcgaagtgaacacccacttgcccagctttactgccattcaatcatggaacaccatcattttgaccagtgcctgatgattcttaatagtccaggcaatcagattctcagtggcctctccattgaagaatataagacctcgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggccaattcaacaacggatagcggaacttgtagcaactgaattttttgatcaaggagacagagagagagaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcaggaatggcaggcccttgcagaacagcag

SEQ ID NO: 37 - DNA, 981 bp, synthetic construct
  misc_feature 1..981: note = source = /note="Description of Artificial Sequence: Synthetic Destabilizing Domain (DD)"
  source 1..981: mol_type = other DNA
atggaaggaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattctatcatggaacaccatcattttgaccagtgcctgatgattcttaatagtccaggcaatcagattctcagtggcctctccattgaagaatataagaccacgttgaaaataatcaagcaagctattttagcaacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagacgccatctgcttgcaactgtatgaggccctgacccacgtgtcagaggacagtttccctttgctagatggctgcagaaagaacaggcagaaatggcaggcccttgcagaacagcag

SEQ ID NO: 38 - DNA, 981 bp, synthetic construct
  misc_feature 1..981: note = source = /note="Description of Artificial Sequence: Synthetic Destabilizing Domain (DD)"
  source 1..981: mol_type = other DNA
atggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagagcttccggatgaaacatgaggttctttgcagatggattttaagtgttaagaagaattatcgggagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgagtaactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgttctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 39 - DNA, 981 bp, synthetic construct
  misc_feature 1..981: note = source = /note="Description of Artificial Sequence: Synthetic Destabilizing Domain (DD)"
  source 1..981: mol_type = other DNA
atggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagccttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgttgttggcaatgctgatgacagcttgtgatctttctgcaattacaaaaccctggcctattcaacaacggatagcggaactcgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 40 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgctagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgcccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcagaaggagttgtttttggcaatgctgatgacagcttgtaatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccatgtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 41 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagcttgactgccattcaatcatggaacaccatcactttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacatataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 42 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatggaacatgaggttctttgcagatggattttaagtgttaagaagaattatcggaagcatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagatactcagtggcctctccattgaagaatataagaccacgttgaaaataatcaagcaagctattttagcaacagacctagcactgtacattaagaggcgaggagaattttttgaacttataagaaaaaatcaattcaatttggaagatcctcatcaaaaggagttgtttttggcaatgctgatgacagcttgtgttctttctgcaattacaaaaccctggcctattcaacaacggatagcggaacttgtagcaactgaattttttgatcaaggagacagagagagaaaagaactcaacatagaacccactgatttaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 43 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtggtctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 44 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcctcagtgactttgagctgtctgatctggaaacagcactgtgtacaattcggatgtttactgacctcaaccttgtgcagaacttccggatgaaacatgaggttctttgcagatggattttaagtgttaaggagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacaaacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaattcaacatagaacccacagatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 45 - DNA, 978 bp, synthetic construct
  misc_feature 1..978: note = source = /note="Description of Artificial Sequence: Synthetic Destabilizing Domain (DD)"
  source 1..978: mol_type = other DNA
gacgaaacaagagagctacagtcgttagcggctgctgtggtgccatctgcccagacccttagaattactgactttagcttcagtgactctgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaagagcaggcaaaattcagaacaagctgactgacctggagatacttgcattgctgattgctgcactaagccacgatttggatcaccgtggtgtgaataactcttacatacagcgaagtgaacatccacttgcccagctttaccgccattcaatcatggaacaccatcattttgaccagtgcctgatgattcttaatagtcccggcaatcagattctcagtggcctctccattgaagaatataagaccacgctgaaaataatcaagcaagctattttagctacagacctagcactgtacattaagaggcgaggagaattttttgaacttataagaaaaagtcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggggacagagagagaaaagagctcaacacagaacccactgatctaatgaacagggagaagaaaaacaaaatcccaagtgcgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 46 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagcatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagcttaactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacatcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 47 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgctagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagagcaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgcccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcagaaggagttgtttttggcaatgctgatgacagcttgtaatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccatgtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 48 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatgggttttaagtgttaagaagaattatcggaagaatgttgcctatcacaattggagacatgcctttaatacagctcagtgcatgtttgccgctctaaaagcaggcaaaattcagaataagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataaggccacgttgaaaataatcaagcaagctattttagctacagacctagcactgtacattaagaggcgaggagaattttttgaacttataagaaaaaatcaactcaattcggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaacttaacatagaacccactgatccaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 49 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttggcttcagtgactttgagctgtctgatctggaaacagcactgtgtacaattcggatgtttactgacctcaaccttgcgcagagcttccggatggaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataacccttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgttcccctttgctagatggctgcagaaagaacaggcagaaatggcaggcccttgcagaacagcag

SEQ ID NO: 50 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatccgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctggttgctgcactaagccacgatttggaccaccgtggtgtgaataactcttacatacagcgaagtgaacatccacttgcccagctttactgccattcaatcatggaacaccatcattttgaccagtgcctgatgattcttaatagtccaggcaatcaaattctcagtggcctctccattgaagaatataagaccacgttgaaaataatcaagcaggctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtacgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 51 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcctcagtgaccttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacaaacctagcactgtacattaagaggcgaggagaattttttgaacttataagaaaaaatcaattcaatttggaagatcctcatcaaaaggagttgtttttggcaatgctgatgacagcttgtgatctttttgcaattacaaaaccctggcctattcaacaacggatagcggaacttgtagcaactgaattttttgatcaaggagacagagagagaaaagaactcaacatagaaccccctgatttaatgaacagggagaagaaaaacaaaattccaagtatgcaagttgggttcatagatgccatctgcttgcaactgtatgaggccctgacccacgtgtcagaggactgtttccctttgctagatggctgcagaaagaacaggcagaaatggcaggcccttgcagagcagcag

SEQ ID NO: 52 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacggtcgttagcggctgctgtggtgccatctgcccagacccttaaaattactgaccttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccgttcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatttaatgaacagggagaagaaaaacaaaatcccaagtatgcaagttgggttcatagatgccacctgcttgcaactgtatgaggccctgacccacgtgtcagaggactgtttccctttgctagatggctgcagaaagaacaggcagaaatggcaggcccttgcagaacagcag

SEQ ID NO: 53 - protein, 326 aa, synthetic construct
  REGION 1..326: note = source = /note="Description of Artificial Sequence: Synthetic Destabilizing Domain (DD)"
EETRELQSLAAAVVPSAQTLKITDFSLSDFELSDLETALCTIRMFTDLNLVQNFRMKHEVLCRWILSVKENYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATNLALYIKRRGEFFELIRKNQFNLEDPHQKELFLAMLMTACNLSAITKPWPIQQRIAELVATEFFDQGDRERKEFNIEPTDLMNREKKNKIPSMQVGFIDAICLQLYEALTHVSEDCFPLLDGCRKNRQKWQALAEQQ

SEQ ID NO: 54 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcctcagtgactttgagctgtctgatctggaaacagcactgtgtacaattcggatgtttactgacctcaaccttgtgcagaacttccggatgaaacatgaggttctttgcagatggattttaagtgttaaggagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacaaacctagcactgtacattaagaggcgaggagaattttttgaacttataagaaaaaatcaattcaatttggaagatcctcatcaaaaggagttgtttttggcaatgctgatgacagcttgtaatctttctgcaattacaaaaccctggcctattcaacaacggatagcggaacttgtagcaactgaattttttgatcaaggagacagagagagaaaagaatttaacatagaacccacagatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 55 - protein, 326 aa, synthetic construct
  REGION 1..326: note = source = /note="Description of Artificial Sequence: Synthetic Destabilizing Domain (DD)"
EETRELQSLAAAVVPSAQTLKITDFSLSDFELSDLETALCTIRMFTDLNLVQNFRMKHEVLCRWILSVKENYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATNLALYIKRRGEFFELIRKNQFNLEDPHQKELFLAMLMTACGLSAITKPWPIQQRIAELVATEFFDQGDRERKEFNIEPTDLMNREKKNKIPSMQVGFIDAICLQLYEALTHVSEDCFPLLDGCRKNRQKWQALAEQQ

SEQ ID NO: 56 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcctcagtgactttgagctgtctgatctggaaacagcactgtgtacaattcggatgtttactgacctcaaccttgtgcagaacttccggatgaaacatgaggttctttgcagatggattttaagtgttaaggagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacaaacctagcactgtacattaagaggcgaggagaattttttgaacttataagaaaaaatcaattcaatttggaagatcctcatcaaaaggagttgtttttggcaatgctgatgacagcttgtggtctttctgcaattacaaaaccctggcctattcaacaacggatagcggaacttgtagcaactgaattttttgatcaaggagacagagagagaaaagaatttaacatagaacccacagatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 57 - protein, 576 aa, synthetic construct
  REGION 1..576: note = source = /note="Description of Artificial Sequence: Synthetic Destabilizing Domain (DD)"
MVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYKEFYPYDVPDYAEETRELQSLAAAVVPSAQTLKITDFSLSDFELSDLETALCTIRMFTDLNLVQNFRMKHEVLCRWILSVKENYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATNLALYIKRRGEFFELIRKNQFNLEDPHQKELFLAMLMTACDLSAITKPWPIQQRIAELVATEFFDQGDRERKEFNIEPTDLMNREKKNKIPSMQVGFIDAICLQLYEALTHVSEDCFPLLDGCRKNRQKWQALAEQQ

SEQ ID NO: 58 - DNA, 1731 bp, synthetic construct
  misc_feature 1..1731: note = source = /note="Description of Artificial Sequence: Synthetic Destabilizing Domain (DD)"
  source 1..1731: mol_type = other DNA
atggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaaggaattctatccgtacgacgtaccagactacgcagaagaaacaagagagctacagtcgttagcggctgctgtggtgccatctgcccagacccttaaaattactgactttagcctcagtgactttgagctgtctgatctggaaacagcactgtgtacaattcggatgtttactgacctcaaccttgtgcagaacttccggatgaaacatgaggttctttgcagatggattttaagtgttaaggagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacaaacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaattcaacatagaacccacagatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcagtga

SEQ ID NO: 59 - protein, 576 aa, synthetic construct
  REGION 1..576: note = source = /note="Description of Artificial Sequence: Synthetic Destabilizing Domain (DD)"
MVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYKEFEETRELQSLAAAVVPSAQTLKITDFSLSDFELSDLETALCTIRMFTDLNLVQNFRMKHEVLCRWILSVKENYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATNLALYIKRRGEFFELIRKNQFNLEDPHQKELFLAMLMTACDLSAITKPWPIQQRIAELVATEFFDQGDRERKEFNIEPTDLMNREKKNKIPSMQVGFIDAICLQLYEALTHVSEDCFPLLDGCRKNRQKWQALAEQQYPYDVPDYA

SEQ ID NO: 60 - DNA, 1731 bp, synthetic construct
  misc_feature 1..1731: note = source = /note="Description of Artificial Sequence: Synthetic Destabilizing Domain (DD)"
  source 1..1731: mol_type = other DNA
atggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaaggaattcgaagaaacaagagagctacagtcgttagcggctgctgtggtgccatctgcccagacccttaaaattactgactttagcctcagtgactttgagctgtctgatctggaaacagcactgtgtacaattcggatgtttactgacctcaaccttgtgcagaacttccggatgaaacatgaggttctttgcagatggattttaagtgttaaggagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacaaacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaattcaacatagaacccacagatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcagtatccgtacgacgtaccagactacgcatga

SEQ ID NO: 61 - DNA, 2502 bp, Homo sapiens
  source 1..2502: mol_type = other DNA
atgttgccctttggagacaaaacaagagaaatggtcaatgcatggtttgctgagagagttcacaccatccctgtgtgcaaggaaggtatcagaggccacaccgaatcttgctcttgtcccttgcagcagagtcctcgtgcagataacagtgcccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactga

SEQ ID NO: 62 - DNA, 2472 bp, Homo sapiens
  source 1..2472: mol_type = other DNA
atggtcaatgcatggtttgctgagagagttcacaccatccctgtgtgcaaggaaggtatcagaggccacaccgaatcttgctcttgtcccttgcagcagagtcctcgtgcagataacagtgcccctggaacaccaaccaggaaaatctctgcctctgaatttgaccggcctcttagacccattgttgtcaaggattctgagggaactgtgagcttcctctctgactcagaaaagaaggaacagatgcctctaacccctccaaggtttgatcatgatgaaggggaccagtgctcaagactcttggaattagtgaaggatatttctagtcatttggatgtcacagccttatgtcacaaaattttcttgcatatccatggactgatatctgctgaccgctattccctgttccttgtctgtgaagacagctccaatgacaagtttcttatcagccgcctctttgatgttgctgaaggttcaacactggaagaagtttcaaataactgtatccgcttagaatggaacaaaggcattgtgggacatgtggcagcgcttggtgagcccttgaacatcaaagatgcatatgaggatcctcggttcaatgcagaagttgaccaaattacaggctacaagacacaaagcattctttgtatgccaattaagaatcatagggaagaggttgttggtgtagcccaggccatcaacaagaaatcaggaaacggtgggacatttactgaaaaagatgaaaaggactttgctgcttatttggcattttgtggtattgttcttcataatgctcagctctatgagacttcactgctggagaacaagagaaatcaggtgctgcttgaccttgctagtttaatttttgaagaacaacaatcattagaagtaattttgaagaaaatagctgccactattatctctttcatgcaagtgcagaaatgcaccattttcatagtggatgaagattgctccgattctttttctagtgtgtttcacatggagtgtgaggaattagaaaaatcatctgatacattaacaagggaacatgatgcaaacaaaatcaattacatgtatgctcagtatgtcaaaaatactatggaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccagatgaaacatgaggttctttgcagatggattttaagtgttaagaagaattatcggaagaatgttgcctatcataattggagacatgcctttaatacagctcagtgcatgtttgctgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcagaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaactga

SEQ ID NO: 63 - DNA, 1023 bp, synthetic construct
  misc_feature 1..1023: note = source = /note="Description of Artificial Sequence: Synthetic Destabilizing Domain (DD)"
  source 1..1023: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaac

SEQ ID NO: 64 - DNA, 1230 bp, synthetic construct
  misc_feature 1..1230: note = source = /note="Description of Artificial Sequence: Synthetic Destabilizing Domain (DD)"
  source 1..1230: mol_type = other DNA
ggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaac

SEQ ID NO: 65 - DNA, 1368 bp, synthetic construct
  misc_feature 1..1368: note = source = /note="Description of Artificial Sequence: Synthetic Destabilizing Domain (DD)"
  source 1..1368: mol_type = other DNA
gaaccacttaatatcccagatgtcagtaaggataaaagatttccctggacaactgaaaatacaggaaatgtaaaccagcagtgcattagaagtttgctttgtacacctataaaaaatggaaagaagaataaagttataggggtttgccaacttgttaataagatggaggagaatactggcaaggttaagcctttcaaccgaaatgacgaacagtttctggaagcttttgtcatcttttgtggcttggggatccagaacacgcagatgtatgaagcagtggagagagccatggccaagcaaatggtcacattggaggttctgtcgtatcatgcttcagcagcagaggaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcaggagaagatgctgattaatggggaaagcggccaggccaagcggaac

SEQ ID NO: 66 - protein, 326 aa, synthetic construct
  REGION 1..326: note = source = /note="Description of Artificial Sequence: Synthetic Destabilizing Domain (DD)"
EETRELQSLAAAVVPSAQTLKITDFSFSDFELSDLETALCTIRMFTDLNLVQNFRMKHEVLCRWILSVKKNYRKNVAYHNWRHAFNTAQCMFAALKAGEIQNKLTDLEILALLIAALSHDLDHRGVNNSYIQRSEHPLAQLYCHSIMEHHHFDQCLMILNSPGNQILSGLSIEEYKTSLKIIKQAILATDLALYIKRRGEFFELIRKNQFNLEDPHQKELFLAMLMTACDLSAITKPWPIQQRIAELVATEFFDQGDREREELNIEPTDLMNREKKNKIPSMQVGFIDAICLQLYEALTHVSEDCFPLLDGCRKNRQEWQALAEQQ

SEQ ID NO: 67 - protein, 326 aa, synthetic construct
  REGION 1..326: note = source = /note="Description of Artificial Sequence: Synthetic Destabilizing Domain (DD)"
EGTRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACDLSAITKPWPIQQRIAELVATEFFDQGDRERKELNIEPTDLMNREKKNKIPSMQVGFIDAICLQLYEALTHVSEDSFPLLDGCRKNRQKWQALAEQQ

SEQ ID NO: 68 - protein, 326 aa, synthetic construct
  REGION 1..326: note = source = /note="Description of Artificial Sequence: Synthetic Destabilizing Domain (DD)"
EETRELQSLAAAVVPSAQTLKITDFSFSDFELSDLETALCTIRMFTDLNLVQSFRMKHEVLCRWILSVKKNYRENVAYHNWRHAFNTAQCMFAALKAGKIQNKLTDLEILALLIAALSHDLDHRGVSNSYIQRSEHPLAQLYCHSIMEHHHFDQCLMILNSPGNQILSGLSIEEYKTTLKIIKQAILATDLALYIKRRGEFFELIRKNQFNLEDPHQKELFLAMLMTACVLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 69 - protein, 326 aa, synthetic construct
  REGION 1..326: note = source = /note="Description of Artificial Sequence: Synthetic Destabilizing Domain (DD)"
EETRELQSLAAAVVPSAQTLKITDFSFSDFELSDLETALCTIRMFTDLNLVQNFRMKHEVLCRWILSVKKNYRKNVAYHNWRHAFNTAQCMFAALKAGKIQNKLTDLEILALLIAALSHDLDHRGVNNSYIQRSEHPLAQPYCHSIMEHHHFDQCLMILNSPGNQILSGLSIEEYKTTLKIIKQAILATDLALYIKRRGEFFELIRKNQFNLEDPHQKELLLAMLMTACDLSAITKPWPIQQRIAELVATEFFDQGDRERKELNIEPTDLMNREKKNKIPSMQVGFIDAICLQLYEALTHVSEDCFPLLDGCRKNRQKWQALAEQQ

SEQ ID NO: 70 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcgaaattcagaacaagctgactgacctggagatacttgcattgctgattgctgcactaagccacgatttggatcaccgtggtgtgaataactcttacatacagcgaagtgaacacccacttgcccagctttactgccattcaatcatggaacaccatcattttgaccagtgcctgatgattcttaatagtccaggcaatcagattctcagtggcctctccattgaagaatataagacctcgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggccaattcaacaacggatagcggaacttgtagcaactgaattttttgatcaaggagacagagagagagaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcaggaatggcaggcccttgcagaacagcag

SEQ ID NO: 71 - DNA, 978 bp, synthetic construct
  misc_feature 1..978: note = source = /note="Description of Artificial Sequence: Synthetic Destabilizing Domain (DD)"
  source 1..978: mol_type = other DNA
gaaggaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagctttactgccattctatcatggaacaccatcattttgaccagtgcctgatgattcttaatagtccaggcaatcagattctcagtggcctctccattgaagaatataagaccacgttgaaaataatcaagcaagctattttagcaacagacctagcactgtacattaagaggcgaggagaattttttgaacttataagaaaaaatcaattcaatttggaagatcctcatcaaaaggagttgtttttggcaatgctgatgacagcttgtgatctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagacgccatctgcttgcaactgtatgaggccctgacccacgtgtcagaggacagtttccctttgctagatggctgcagaaagaacaggcagaaatggcaggcccttgcagaacagcag

SEQ ID NO: 72 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagagcttccggatgaaacatgaggttctttgcagatggattttaagtgttaagaagaattatcgggagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgagtaactcttacatacagcgaagtgaacatccacttgcccagctttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgtttttggcaatgctgatgacagcttgtgttctttctgcaattacaaaaccctggcctattcaacaacggatagcggaacttgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 73 - DNA, 978 bp, synthetic construct
  misc_feature 1..978: note = source = /note="Description of Artificial Sequence: Synthetic Destabilizing Domain (DD)"
  source 1..978: mol_type = other DNA
gaagaaacaagagagctacagtcgttagcggctgctgtggtgccatctgcccagacccttaaaattactgactttagcttcagtgactttgagctgtctgatctggaaacagcactgtgtacaattcggatgtttactgacctcaaccttgtgcagaacttccggatgaaacatgaggttctttgcagatggattttaagtgttaagaagaattatcggaagaatgttgcctatcataattggagacatgcctttaatacagctcagtgcatgtttgccgctctaaaagcaggcaaaattcagaacaagctgactgacctggagatacttgcattgctgattgctgcactaagccacgatttggatcaccgtggtgtgaataactcttacatacagcgaagtgaacatccacttgcccagccttactgccattcaatcatggaacaccatcattttgaccagtgcctgatgattcttaatagtccaggcaatcagattctcagtggcctctccattgaagaatataagaccacgttgaaaataatcaagcaagctattttagctacagacctagcactgtacattaagaggcgaggagaattttttgaacttataagaaaaaatcaattcaatttggaagatcctcatcaaaaggagttgttgttggcaatgctgatgacagcttgtgatctttctgcaattacaaaaccctggcctattcaacaacggatagcggaactcgtagcaactgaattttttgatcaaggagacagagagagaaaagaactcaacatagaacccactgatctaatgaacagggagaagaaaaacaaaatcccaagtatgcaagttgggttcatagatgccatctgcttgcaactgtatgaggccctgacccacgtgtcagaggactgtttccctttgctagatggctgcagaaagaacaggcagaaatggcaggcccttgcagaacagcag

SEQ ID NO: 74 - protein, 9 aa, synthetic construct
  REGION 1..9: note = source = /note="Description of Artificial Sequence: Synthetic Tag"
YPYDVPDYA

SEQ ID NO: 75 - DNA, 27 bp, synthetic construct
  misc_feature 1..27: note = source = /note="Description of Artificial Sequence: Synthetic Tag"
  source 1..27: mol_type = other DNA
tatccgtacgacgtaccagactacgca

SEQ ID NO: 76 - protein, 239 aa, synthetic construct
  REGION 1..239: note = source = /note="Description of Artificial Sequence: Synthetic POI sequence"
MVSKGAELFTGIVPILIELNGDVNGHKFSVSGEGEGDATYGKLTLKFICTTGKLPVPWPTLVTTLSYGVQCFSRYPDHMKQHDFFKSAMPEGYIQERTIFFEDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSALSKDPNEKRDHMIYFGFVTAAAITHGMDELYK

SEQ ID NO: 77 - DNA, 717 bp, synthetic construct
  misc_feature 1..717: note = source = /note="Description of Artificial Sequence: Synthetic POI sequence"
  source 1..717: mol_type = other DNA
atggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaag